(19) United States
(12) Patent Application Publication
(10) Pub. No.: US 2026/0363577 A1
(43) Pub. Date: Nov. 19, 2026

(54) SYSTEMS AND METHODS FOR MULTIMODAL BIOMECHANICAL MOVEMENT ANALYSIS

(71) Applicant: REHABILITATION INSTITUTE OF CHICAGO, Chicago, IL (US)

(72) Inventor: Ronald James Cotton, Chicago, IL (US)

(73) Assignee: REHABILITATION INSTITUTE OF CHICAGO, Chicago, IL (US)

(21) Appl. No.: 19/655,315

(22) Filed: Apr. 22, 2026

Related U.S. Application Data
(60) Provisional application No. 63/792,644, filed on Apr. 22, 2025.
(60) Provisional application No. 63/793,236, filed on Apr. 23, 2025.

(51) Int. Cl. A61B 5/11; A61B 5/00; G06N 3/045; G06N 3/08

(52) U.S. Cl. CPC A61B 5/112; A61B 5/1128; A61B 5/6802; G06N 3/045; G06N 3/08

(57) ABSTRACT

Systems and methods for analyzing movement of at least one user are provided to perform: receiving kinematic movement data derived from one or more of: video data, sensor data, or biomechanical models; encoding the kinematic movement data into a learned multimodal representation using a foundation model; and performing, using the learned multimodal representation, one or more downstream tasks by decoding the learned multimodal representation using one or more task-specific decoder.

### CROSS-REFERENCE TO RELATED APPLICATIONS

The present application claims the benefit of U.S. App. No. 63/792,644, filed Apr. 22, 2025, and U.S. App. No. 63/793,236, filed Apr. 23, 2025, the entireties of which are incorporated herein by reference for all purposes.

### GOVERNMENT SUPPORT

This invention was made with government support under Award No. R01HD114776 awarded by the National Institutes of Health (NIH). The government has certain rights in the invention.

### FIELD

The present disclosure relates to systems and methods for analyzing human movement. More particularly, it relates to systems and methods for performing clinical gait analysis for treatment or therapy.

### BACKGROUND

Gait disorders occur in many conditions and may reduce independence and quality of life, while increasing the likelihood of complications such as falls. Improving gait is a cornerstone of rehabilitation. Early detection and intervention of gait impairments can reduce falls and their associated morbidity and mortality.

Despite the importance of gait, commonly used clinical measures do not capture the quality of walking. For example, the popular 10-meter walk or 6-minute-walk tests only capture speed and endurance. Other performance-based outcomes like the timed-up-and-go, Berg balance scale, and Functional Gait Assessment indirectly capture stability and functional mobility. The need for more precise measures of gait impairments has been identified for a range of diagnoses including cerebral palsy, multiple sclerosis, stroke, Parkinson's disease, spinal cord injury and in falls risk prediction. Physical therapists favorably view access to gait analysis. A survey of physical therapists identified numerous gait parameters, including walking speed, cadence, step length, toe clearance, hip extension, and joint kinematics (angle, velocity) that they believed would allow them to provide rehabilitation care.

Thus, quantitative analysis of human movement is fundamental in clinical rehabilitation, biomechanics, and mobility assessment. However, clinicians remain hindered by a lack of clinically accessible tools to measure and analyze gait quantitatively. Although advances in computer vision and artificial intelligence have improved data acquisition for movement analysis, including marker-less motion capture using video and human pose estimation (HPE), its impact on clinical care has been minimal, and it has not yet provided a solution to the precise measurement of gait impairments. Obtaining high-quality kinematics in the clinic may be a barrier in certain situations. Interpreting movement data also remains a barrier to clinical impact, particularly for clinicians who lack specialized biomechanics training. It is unrealistic to expect every clinician to become an expert in analyzing raw biomechanical data. Therefore, improving health and mobility outcomes through widespread access to gait analysis depends on scalable, automated, and reliable analytics.

While existing machine learning systems can solve isolated tasks (e.g., pose estimation or activity recognition), they fail to generalize to impaired or clinical populations, poorly localize activities within a video (e.g., identifying when a subject is walking or completing a task used in a clinical assessment), lack biomechanical grounding, and do not support intuitive, natural language interaction. Separately, large language models have demonstrated success in multimodal reasoning but are not designed to understand time-varying biomechanical motion, forces, or clinically meaningful movement quality.

Accordingly, there exists a need for integrated systems that (i) learn generalizable representations of movement, (ii) infer clinically significant biomechanical quantities, and (iii) provide natural language interfaces for querying and interpreting movement data.

### SUMMARY

The present invention provides systems and methods for multimodal biomechanical movement analysis using a foundation model trained on large-scale movement data and a language-based interface. Particularly, the systems and methods include multimodal foundation models that integrate biomechanical representations of movement with machine learning and natural language processing to perform clinical gait analysis, activity recognition, kinetic inference, and clinician-facing language interaction.

In certain embodiments, the system: receives kinematic movement data derived from video, sensor data, and/or biomechanical models; encodes the movement into a learned multimodal representation using a foundation model; performs one or more downstream tasks, including: activity recognition, temporal localization, gait event detection, inference of joint torques and ground reaction forces, assessment of movement quality; and interfaces with a language model that enables natural language queries and responses that are clinically relevant and grounded in biomechanical representations of motion.

In some embodiments, movement is tokenized into discrete representations that can be processed by a language model.

The invention enables scalable, clinically meaningful movement analytics and improves accessibility of gait analysis across healthcare and other domains.

### BRIEF DESCRIPTION OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a schematic drawing of an exemplary foundation model system for gait analysis according to embodiments disclosed herein.

FIG. 2 is a flow diagram of an exemplary pipeline for multi-view marker-less motion capture according to embodiments disclosed herein.

FIGS. 3A and 3B are exemplary interfaces showing video data with a virtual skeletal reconstruction of the patient superimposed and a set of graphs showing measurements associated with the hip and knee at different stages (early stage/near admission and later stage/pre-discharge).

FIG. 4A is video capture image of a patient at different stages of motion.

FIG. 4B is a virtual skeletal reconstruction of the patient at different stages of motion.

FIG. 4C is a simplified virtual representation that tracks the position of certain body parts of the patient.

FIG. 4D is a graph showing the relative positions of the certain body parts of the patient at different times.

FIG. 5A is a schematic diagram for a contrastive learning model of gait patterns using temporal crops according to embodiments disclosed herein.

FIG. 5B shows an example of embedding similarity matrices between individual trials, the subject ID with the block diagonal structure indicating trials with the same participant, and the diagnosis associated therewith, according to embodiments disclosed herein.

FIG. 5C is a graph showing exemplary changes over time in the average similarity to control values for a plurality of self-supervised learning (SSL) scores.

FIG. 6 is an exemplary user interface for user annotation according to embodiments disclosed herein.

FIGS. 7A through 7C are schematic diagrams showing visualization of some of the key losses that will be combined for training the foundation model according to embodiments disclosed herein.

FIG. 7D is a schematic diagram of an exemplary foundation model according to embodiments disclosed herein.

FIGS. 8A through 8D show examples of images (video capture images) of patients in motion which may be miscategorized by artificial intelligence.

FIGS. 9A and 9B are exemplary interfaces showing video data with a virtual skeletal reconstruction of the patient superimposed and a set of graphs showing measurements associated with the hip and knee angles for instances of right early swing and left early swing.

FIGS. 10A through 10C are schematic diagrams of various examples of a machine learning model according to embodiments disclosed herein.

FIG. 11 is an exemplary user interface for use in a biomechanics large language model (LLM) according to embodiments disclosed herein.

FIGS. 12A through 12F are exemplary confusion matrices used for analyzing the foundation model according to embodiments disclosed herein.

FIG. 13 is a flow diagram for a method of using and training a foundation model according to embodiments disclosed herein.

### DETAILED DESCRIPTION

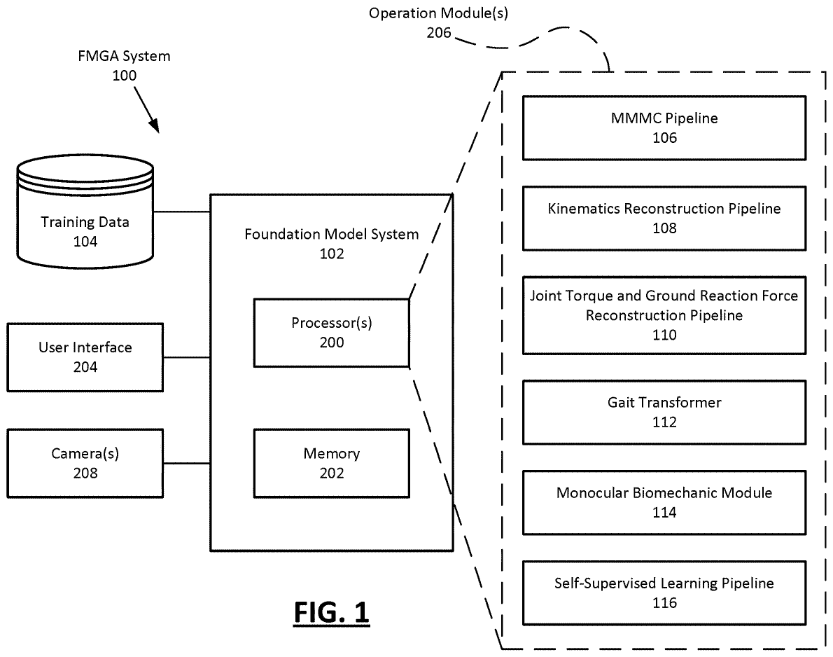

The present invention includes a trained system for Foundation Model for Gait Analysis (FMGA), along with a natural language interface that can provide scalable interpretation of movement data. FIG. 1 shows a schematic diagram of an exemplary FMGA system 100 that includes, for example, a foundation model system 102, a database 104 containing training data to be used by the foundation model system 102, and a user interface 204. The system 102 may include one or more processing units or processors 200 and at least one memory unit 202 such as storage module or a non-transitory computer-readable medium storing instructions thereon that is accessible by the processors 200 to perform one or more processes or algorithms as disclosed herein. For example, the processors 200 may include one or more processors located at a single location or a plurality of locations that are separated from each other, such as a network of remotely located processors. The network may include, for example, a cloud computing network or a server system that operably connects together the plurality of processors and/or the plurality of memory devices so as to increase the computing power for performing the processes or algorithms. The user interface 204 may include one or more graphical user interfaces (GUIs) or display devices (e.g., computer screen, tablet or phone touchscreen, medical computer display, etc.) that is capable of providing information to be accessible by one or more users, such as physicians, therapists, and/or patients, as well as to receive inputs and/or feedbacks from the users, such as for further training of the foundation model system 102 as further disclosed herein.

The processor 200 may include, implement, or be operably coupled with one or more operation modules 206, such as those including but not limited to one or more of: a Multi-view Marker-less Motion Capture (MMMC) pipeline 106, a kinematics reconstruction pipeline 108, a joint torque and ground reaction force reconstruction pipeline 110, a gait transformer 112, a monocular biomechanic module 114, and a self-supervised learning pipeline 116. It is to be understood that in some examples, a single module or device may include functionalities associated with one or more of the operation modules 206, and in some examples, a plurality of modules or devices may cover the functionalities associated with a single operation module 206. In some examples, one or more of the operation modules 206 may be incorporated within another operation module 206 as explained herein. The detailed functionalities associated with each operation module 206 will be explained herein.

### Foundation Model for Gait Analysis (FMGA)

The FMGA system 100 shown in FIG. 1 and disclosed herein includes a multimodal non-language-based foundation model for understanding human movement. A foundation model is a large-scale AI model trained on a vast, diverse dataset (often using self-supervised learning or SSL) that can be adapted to a wide variety of downstream tasks, acting as a versatile “foundation” for applications in generative artificial intelligence. Outputs of the system may include movement, action, activities, and movement quality. The model is trained on human movement data representing diverse activities and populations, including individuals with movement impairments.

In one aspect, the FMGA system 100 computes kinetics, i.e., joint torques and ground reaction forces, from the observed kinematics. Measuring these forces allows for obtaining a deeper understanding of the gait impairments of individuals. In addition, the FMGA system 100 measures ground reaction forces and understands how the foot makes contact with the ground relative to the rest of the body. This important task can guide the prescription of orthoses. Furthermore, tracking foot ground contacts makes it possible to divide gait into cycles and measure spatiotemporal gait parameters.

In another aspect, the FMGA system 100 is also trained to infer joint torques and ground reaction forces from kinematics by incorporating self-supervised learning with biomechanical physics simulation into the training process. The system may use any high-performance, graphics processing unit (GPU)-accelerated, differentiable biomechanical simulator (e.g., MuJoCo which is an advanced physics simulation engine, and MyoSkeleton which is a universal human skeletal model), to power these methods.

In addition to kinetics, the FMGA system 100 may identify the activities performed in a video clip and localize when they are occurring. This is key to clinical translation, where capturing unstructured videos or diverse activities is typical, and analytics first requires identifying the segment to analyze. Existing algorithms for activity recognition do not generalize to clinical populations; for example, they rarely are capable of identifying walking for people with severe gait impairments moving slowly. They also cannot recognize common clinical outcome assessments, such as the timed-up-and-go (TUG). The FMGA system 100 is trained on trials of clinical activities with clip-level annotations of the activity being performed by people with movement impairments, in addition to weakly supervised activity localization to use these clip-level annotations and learn to recognize and localize the activities.

Further, the FMGA system 100 is configured to capture the holistic aspects of movement and its quality, akin to how a clinician looks at a patient moving for an overall impression of health. Self-supervised learning from a movement dataset produces a gait representation containing useful clinical information, such as diagnosis. This gait representation has a meaningful topology, and people with a recent stroke undergoing inpatient rehabilitation moved in this embedding space towards the able-bodied cluster.

The FMGA system 100 described herein incorporates several improvements to the performance and accessibility of video-based gait analysis as explained herein, including, for example by virtue of the one or more operation modules 206.

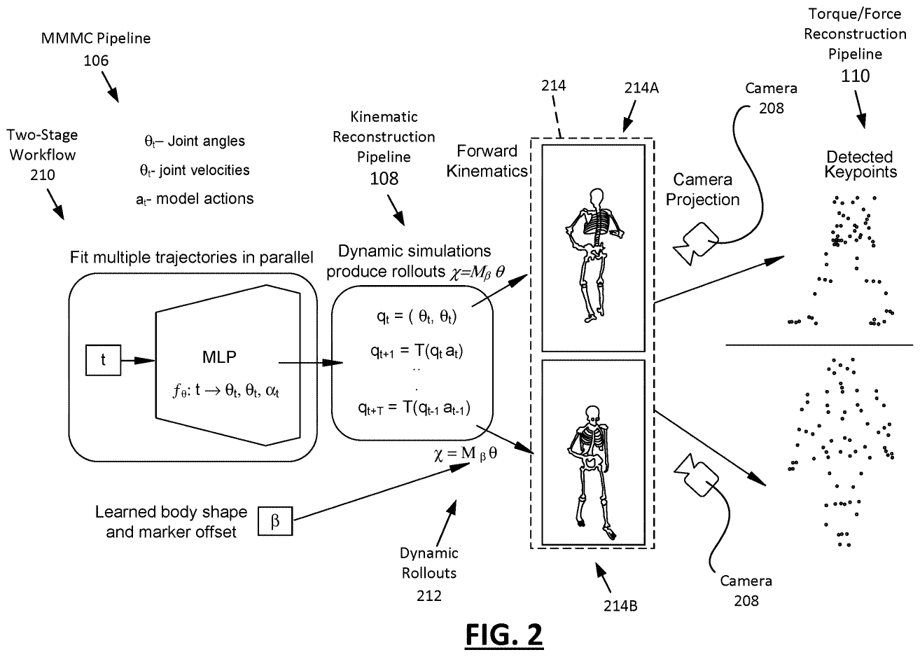

Multi-view Marker-less Motion Capture (MMMC) Pipeline 106: The MMMC pipelines 106 using multiple cameras (such as cameras 208 shown in FIGS. 1 and 2) for biomechanical analysis has shown utility and validity for gait. An MMMC pipeline 106 may follow a two-stage workflow 210 that mimics marker-based methods: first, triangulate virtual marker trajectories from key points detected on different views and then fit a biomechanical model to these trajectories. The workflow 210 may incorporate a Multilayer Perceptron (MLP) or any other suitable foundational feedforward artificial neural network. This two-stage approach prevents biomechanical constraints and scaling from being incorporated into estimating the virtual 3D marker locations. However, in some examples, for purposes of the FMGA system 100, this may be suboptimal if the detection accuracy of computer vision key points is noisier when compared to optical marker locations, which are tracked very precisely. Another possible limitation of existing systems may be that they are rarely validated on clinical populations. Many pretrained algorithms do not generalize to clinical subjects who may be underrepresented in public training datasets.

To address such potential limitations, the FMGA system 100 incorporates an optimized pipeline for biomechanical analysis using MMMC, that is, the MMMC pipeline 106. The approach was validated on a diverse clinical population, finding spatial errors on the order of 1 cm at room-scale tracking. Different key point detectors were compared and found that an algorithm that trains a ‘Rosetta stone’ to learn from a plurality of 3D key point datasets is both accurate and also produces a dense set of key points that are sufficient for high-quality biomechanics. This algorithm contrasts with the vast majority of HPE algorithms that produce a sparse set of key points over the torso, which results in very unstable fits for the hip.

Kinematics Reconstruction Pipeline 108: The FMGA system 100 also incorporates differentiable biomechanics for end-to-end kinematics, such as in a reconstruction pipeline 108 that computes the kinematics fits with an end-to-end optimization that minimizes the error between the detected and predicted key points from the biomechanical trajectories. For example, the kinematics fits may be represented as a virtual skeletal reconstruction 214. This end-to-end approach better handles noise in the detected key points. The system may utilize a high-performance, GPU-accelerated, differentiable biomechanical simulator, such as MuJoCo or MyoSkeleton. GPU acceleration that supports biomechanical models enables a wide range of options to integrate machine-learning approaches to biomechanical analysis. The present system represents the trajectory over each trial as an implicit function implemented with a learned neural network, which provides better learning properties than an explicit representation of the trajectory. This also facilitates taking the derivative with respect to time to obtain joint velocities. The end-to-end optimization approach works particularly well for MMMC, as it can better balance biomechanical constraints, visual uncertainty, and the geometric consistency between views. It allows for jointly optimizing the body scale while solving for the inverse kinematics on multiple trials, avoiding the need for a traditional camera calibration step. This improves the workflow for collecting data in the clinic and is helpful for people who cannot perform standard calibration poses.

Joint Torques and Ground Reaction Force Reconstruction Pipeline 110: In addition to kinematics, the FMGA system 100 simultaneously reconstructs joint torques and ground reaction forces along with the joint angles and joint velocities by extending the implicit representation to include actions. The implicit representation is sampled at arbitrary times, including multiple between-the-frame times. Joint angles and velocities are used to create an initial pose. Then, actions from the implicit representation at future time points are used to create dynamic rollouts 212 using the physics simulator (e.g., MuJoCo). These rollouts 212 are optimized to match the future detected key points, which jointly fit the kinematics, torques, and ground reaction forces from MMMC data (FIG. 2).

Specifically, FIG. 2 is an overview of end-to-end kinetic reconstruction of multi-view marker-less motion capture data, which uses an implicit representation of the poses and actions and directly optimizes the dynamic rollouts. For example, the two poses (top pose shown as virtual skeletal reconstruction 214A and bottom pose shown as virtual skeletal reconstruction 214B) show the beginning and end of a mini-rollout during a walking segment. The predicted marker locations are computed for each timepoint of the rollout and compared to the measured key points, and then optimized end-to-end to produce dynamics consistent with the videos.

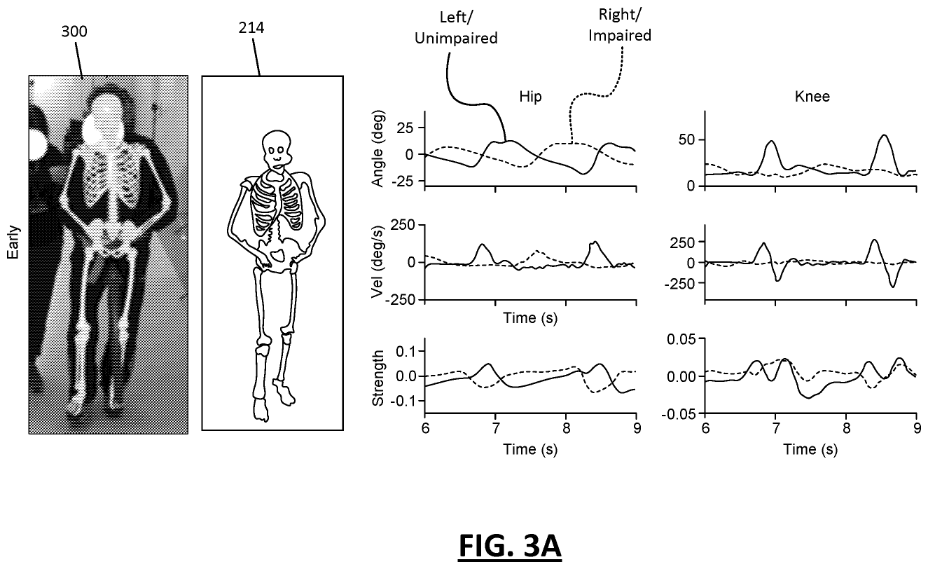

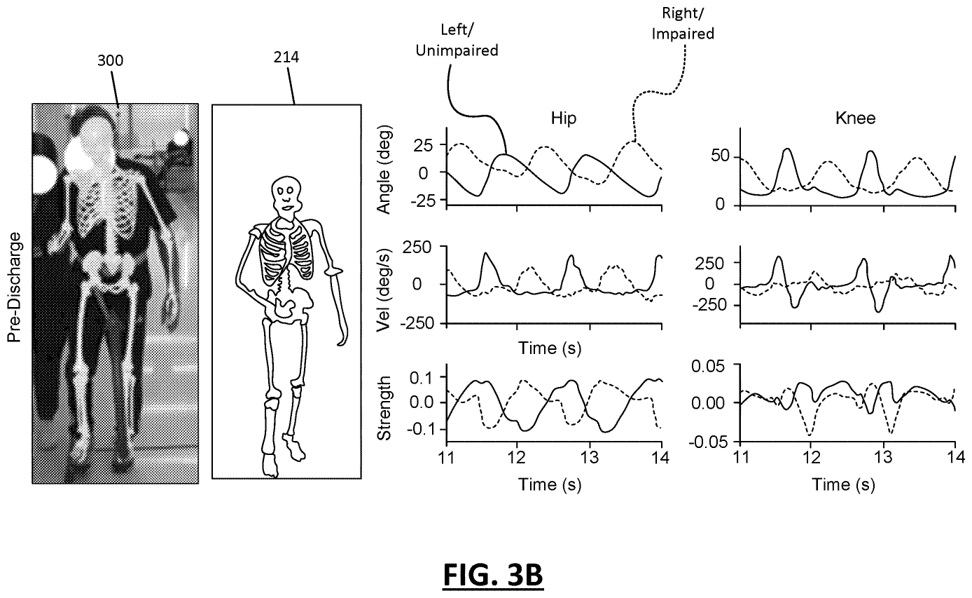

Gait data has been collected from 520 inpatient rehabilitation patients in 718 sessions comprising 8200 trials using this system. This totals 45 hours of multi-view recordings (460 raw hours of video). Many of these participants have longitudinal assessments as they go through inpatient rehabilitation. FIGS. 3A and 3B show an example patient with stroke showing the results of longitudinal gait analysis performed during inpatient rehabilitation. Specifically, FIG. 3A shows information associated with the patient near admission, and FIG. 3B shows information associated with the patient near discharge. In each of FIGS. 3A and 3B, the different lines in the generated graphs (e.g., angle vs time, velocity vs time, and strength vs time, as labeled) show the information associated with the different sides of the patient depending on whether the side has been impaired by stroke; for example, one line shows the left (unimpaired) side, and the other line shows the right (impaired) side for this particular patient. Not only are improvements in the knee flexion kinematics visible, but improvements in control of torque in the hemiparetic knee and hip are visible. With increased hip range during recovery, asymmetrical hip flexion becomes more pronounced. In some examples, the virtual skeletal reconstruction 214 may be provided to visually represent the left and right sides of the body in motion, and the reconstruction 214 may be superimposed on video data 300 of the patient to be shown to the user. This may be used to allow the user to train the system to correct any inaccuracies associated with the reconstruction 214 by comparing it with the actual video of the patient in question.

In summary, FIGS. 3A and 3B show an example of kinematic and kinetic changes observed in the gait of a participant with a recent stroke gait after several weeks of inpatient rehabilitation. This shows dramatic improvements in hip torque symmetry and smaller but substantial improvements in the swing phase knee flexion torque. Routine outcome measures, such as the 10-meter walk test speed, would not capture these changes. Use of monocular video obtained from a smartphone may overcome the time burden to optimize the trajectory, requirement of multiple cameras, and sensitivity to errors when estimating the floor plane, associated with use of this end-to-end kinetic reconstruction.

Gait Transformer 112: A challenge when performing gait analysis using existing key point detectors is identifying the gait cycles. To overcome this, the FMGA system 100 incorporates a trained algorithm to map the outputs from HPE algorithms applied to this data to spatiotemporal gait parameters. This “Gait Transformer” 112 was trained on a large dataset from 934 clinical gait laboratory subjects over 1262 sessions, with nearly 9000 trials of paired video with motion capture data. In operation in the clinic, this utilized a smartphone application that acquires RGB videos, depth videos, and synchronized data from a wearable sensor platform, called the Portable Biomechanics Laboratory (PBL). For example, the PBL may be the database 104 that supplies the training data. The PBL also records the movement data from the phone itself. This system can obtain movement data from a diverse range of patients and has been used to collect data from 340 participants, totaling 78 hours of video. The PBL was also used to prospectively validate the Gait Transformer 112, using manual annotation of walking speeds and the sensor data to detect gait events. The algorithm has the capability of detecting gait events and walking speed when validated in an outpatient rehabilitation clinic.

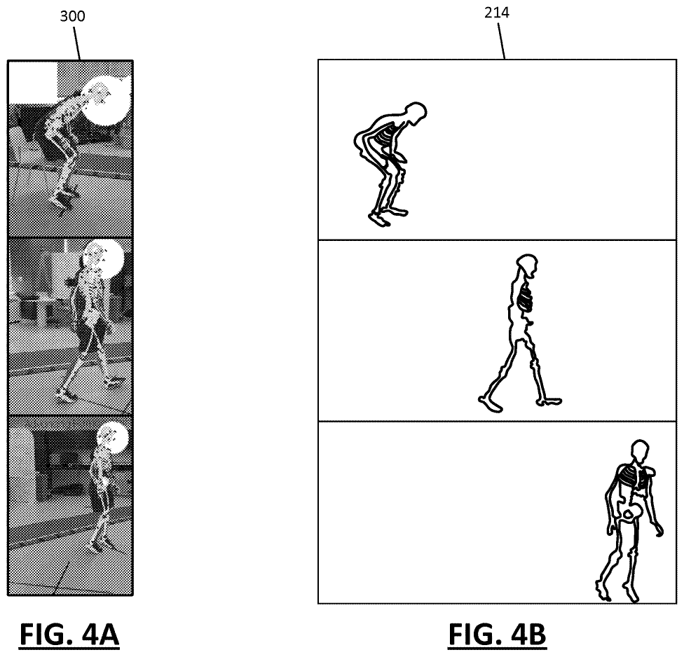

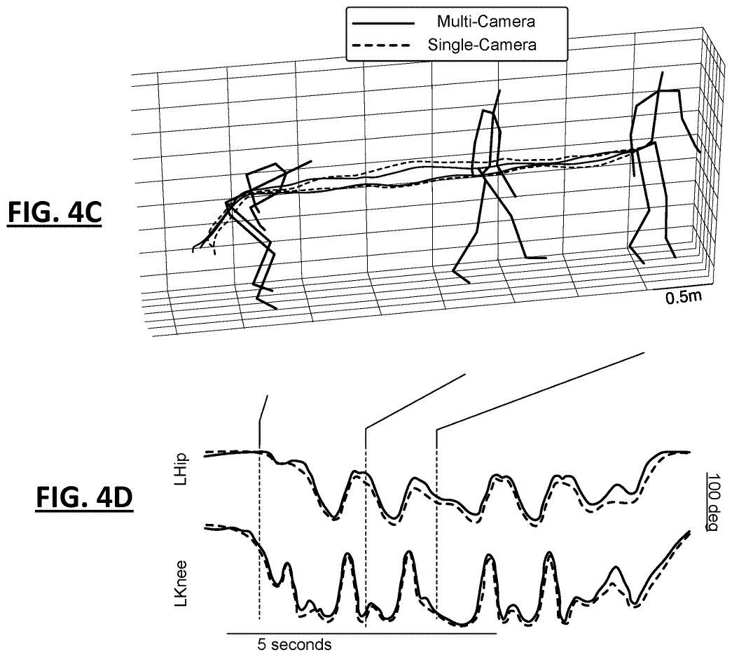

Monocular Biomechanic Module 114: To further improve biomechanical kinematic estimates, the end-to-end optimization approach is applied to video data 300 acquired with the database 104, or the PBL. This method delivers high-quality kinematic trajectories, with sagittal plane hip and knee angles typically having less than 5 degrees of root mean squared errors. This algorithm works for diverse populations, including able-bodied controls, people with a history of stroke, and lower-limb prosthesis users. This method can also disentangle the rotations of a smartphone from the rotation of the person, allowing to accurately track the participant in the world (see, for example, FIGS. 4A through 4D). Absolute tracking accuracy is typically within 6 cm of the tracking from the multicamera system. Thus, this improved method obtains biomechanical fits in a consistent format, as the MMMC pipeline 106 uses only a smartphone. For example, in FIG. 4A, a handheld camera may be used to shoot video data 300 of the patient, and in FIG. 4B, the camera and body orientation changes are disentangled to accurately track a timed-up-and-go test in global space. FIG. 4C shows a simplified virtual representation that tracks the position of the body part, such as the left knee and the left hip in this particular patient, based on whether the video information is provided from a single camera or multiple cameras. FIG. 4D shows the joint kinematics being validated against a multi-camera system for finding close agreement between the two methods.

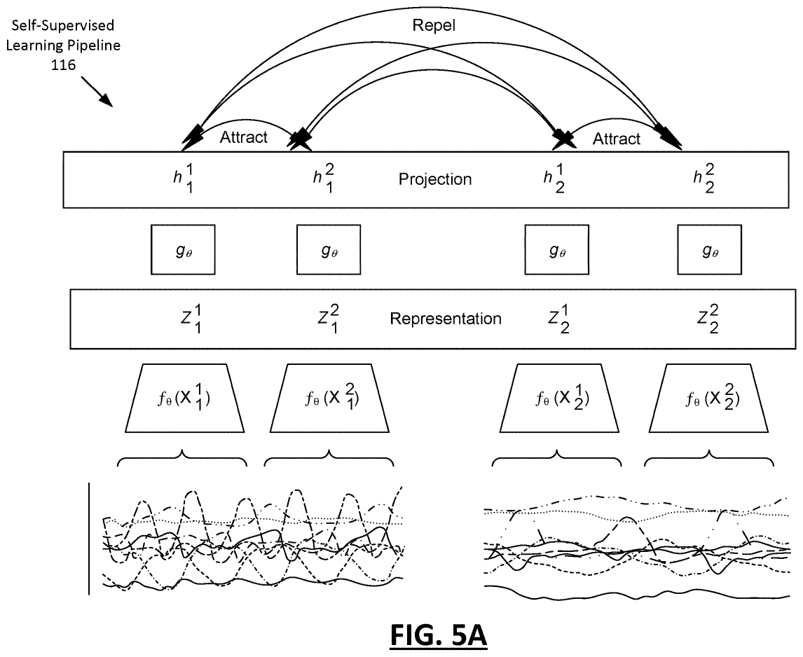

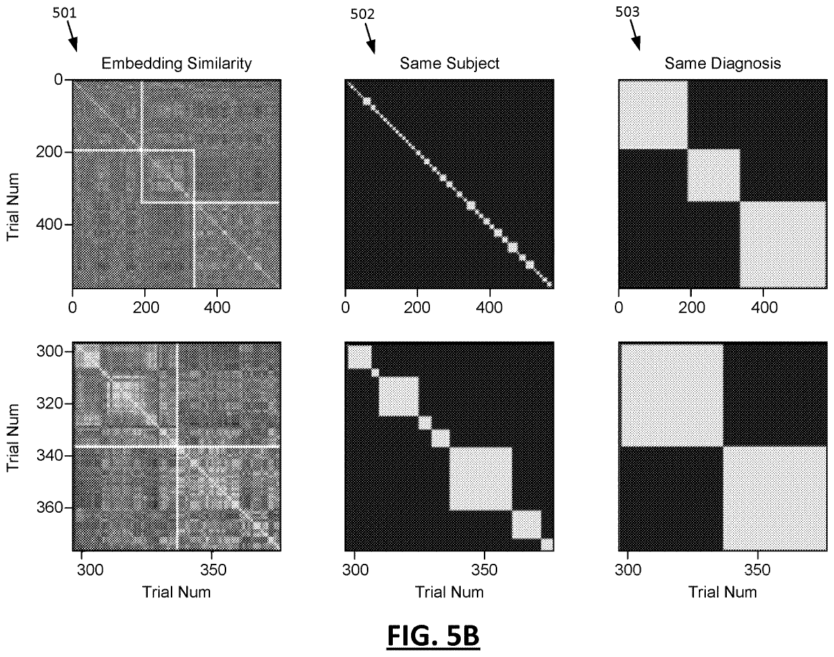

Self-Supervised Learning Pipeline 116: Self-supervised learning (SSL) is used on gait datasets (e.g., gait-based biomarkers) to learn a representation of gait patterns that contains clinically meaningful information. When linked to the framework of biomarkers, such as those provided by the National Institutes of Health (NIH) and/or the U.S. Food and Drug Administration (FDA), it allows for the classification of subjects based on their diagnosis (diagnostic biomarker) and measurement of changes during inpatient rehabilitation (response biomarker). A combination of contrastive learning with temporal crops from a single gait trial was used as positive examples and crops from other trials as negative examples. Despite never being exposed to labels during training, the linear classifier trained on the SSL-learned representation could correctly decode whether a subject had a history of stroke about 90% accurately from only 3 seconds of gait data. Furthermore, the gait representation of subjects with a history of stroke shifted towards the average representation of able-bodied individuals during inpatient rehabilitation (see for example FIGS. 5A through 5C). Combining a next-sample prediction loss as an additional SSL feature further improved performance.

For example, FIGS. 5A through 5C show an example of the SSL training performed by the system for gait bio-makers. FIG. 5A is an overview of contrastive learning of gait patterns using temporal crops. Representation (Z) is the output of the encoder network, f_{θ}(x). Projection (h) is the output of a projection head (g_{θ}) which maps the representation into a different latent space where the contrastive loss is calculated. FIG. 5B shows an example of embedding similarity matrices between individual trials in left column 501. The middle column 502 shows the subject ID, with the block diagonal structure indicating trials with the same participant. The right column 503 shows the diagnosis (prosthesis user, stroke, or able-bodied). In each column the bottom row zooms in on several of the trials to show how the block diagonal structure of the embeddings captures both patterns. FIG. 5C shows the longitudinal changes in the SSL score over time (from left to right) for individuals with stroke during inpatient rehabilitation shifting towards the embedding for able-bodied controls. In FIG. 5C, in all but two instances (labeled as Exception A and Exception B), the average similarity to control increased over time.

These improvements embodied in the operation modules 206 allow for obtaining high-quality kinematic fits to a biomechanically accurate model from both multi-view and monocular videos. The end-to-end optimization method is extended using an implicit representation to obtain kinetics. Use of the Gait Transformer demonstrates that supervised learning from a dataset from a clinical gait laboratory can be used to detect gait event timing from videos. Finally, use of gait-based biomarkers demonstrates that self-supervised learning on large clinical movement datasets can extract clinically meaningful features of movement.

However, implementation of these as separate models has at least two downsides: first, it makes connecting them into practical workflows more cumbersome. More importantly, training them separately does not allow positive transfer between the tasks. Positive transfer is when training on task A in addition to B improves the performance on task B and is a common observation in foundation models. For example, training a combined model to have a deeper understanding of movement forces and gait event timing should improve the assessment of movement quality.

Furthermore, several clinically important tasks are not solved by any existing models that the FMGA system 100 addresses. The first is activity recognition. These algorithms perform poorly in clinical populations where, for example, walking may be much slower and more impaired than what exists in the training data. Additionally, for clinical use, a need exists for models that perform action localization (i.e., identify the start and stop times of actions) in addition to the activity class. The models should also handle the hierarchical nature of a plurality of movement tasks, such as the commonly used Timed-up-and-Go test, composed of the sequence of sitting in a chair, standing, initiating walking, steady-state walking, turning, steady-state walking, stopping, turning, and then sitting. This fine-grained action localization enables quantification of a plurality of therapeutic tasks that involve repetitive movements, such as the 30-second sit-to-stand test. Existing algorithms also perform poorly on clinically relevant activity recognition and localization due to the absence of datasets containing annotations of the movements important to clinicians or impaired demonstrations.

Second, the FMGA system 100 addresses the additional important task of determining the torques and ground reaction forces associated with movements observed from kinematics alone. Ground reaction forces (GRFs) subsume the gait events outputs from the Gait Transformer while capturing the full 3-dimensional force vector and center of pressure from each foot contact with the floor. This is a clinically meaningful measure that is typically only available during expensive gait laboratory assessments. For example, GRFs can help measure the propulsion asymmetry after a stroke, which can predict response to specific interventions. GRFs are also important when optimizing orthotics, such as ensuring that an ankle foot orthoses move the ground reaction force vector behind the knee to reduce hyperextension during loading for people with a hemiparetic gait. In a typical gait laboratory, GRFs allow computing joint torques, which provide important clinical insights, such as the degree of control over muscles in neurologic conditions. The end-to-end kinetic reconstruction method (FIG. 2) shows that it can detect clinically meaningful changes in joint torques during walking throughout inpatient rehabilitation but is too computationally expensive for routine use. The FMGA system 100 also incorporates the contrastive loss from the SSL biomarker method, treating different snippets from within a trial as positive examples and samples from different trials as negative examples. This produces a representation of the movement in a clip that contains clinically meaningful information that can be decoded with task-specific decoders, like decoding diagnosis.

Building on this, the FMGA system 100 is configured to receive kinematic observations of movement from video and perform the following tasks: 1) compute torques and GRFs, 2) detect gait events, 3) perform granular activity recognition and localization, and 4) output a self-supervised representation of movement quality (following the SSL biomarker methodology). As compared to the individual task-specific models described above, the FMGA system 100 achieves positive transfer learning when combining these tasks into a unified model.

### Training Datasets

The model of the FMGA system 100 is trained on various datasets from the training database 104 as explained herein.

Multi-view marker-less motion capture dataset: The MMMC pipeline 106 is used to characterize the gait of individuals seen in inpatient and outpatient rehabilitation clinics. These datasets contain 8 to 12 synchronized camera views, from which the 3D joint trajectories are reconstructed and inverse kinematics fits are performed with a biomechanical model. This data heavily emphasizes gait, including variations in walking speed, tandem gait, functional assessments like timed-up-and-go and the four-square step test, and postural sway testing while standing still. It currently contains 8271 trials from 528 individuals, most of whom are clinical participants. This includes a mixture of data subjects with stroke, spinal cord injury, traumatic brain injury, neuropathy, lower limb prosthesis users, and pediatric participants. In addition to the fixed camera views, many of these trials include data collected simultaneously from the Portable Biomechanics Laboratory, providing a moving camera view, depth imaging, and inertial measurements from wearable sensors. More than 1500 walking trials are also annotated with spatiotemporal parameters from an instrumented walkway, which also can identify the transition from standing to walking to standing. It also includes data for functional tasks like timed-up-and-go, four-square step test, and sway when standing still.

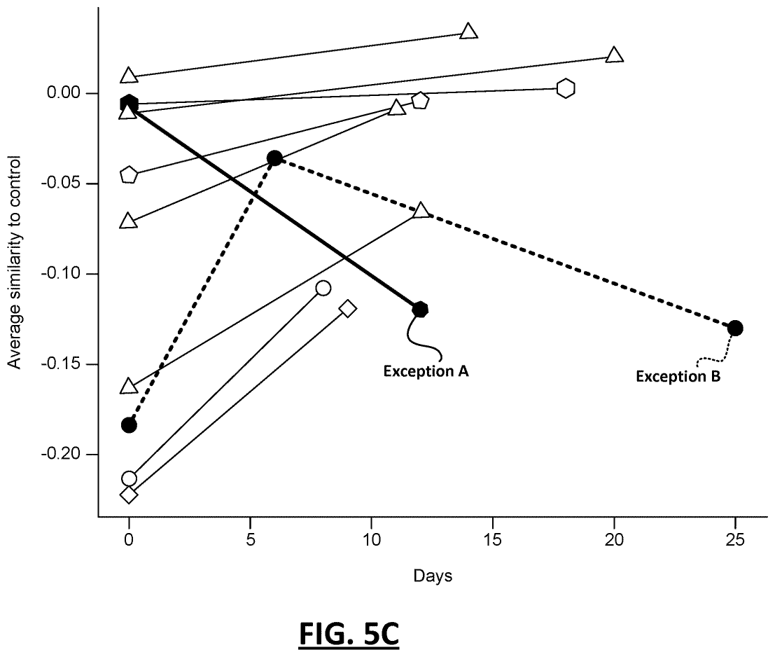

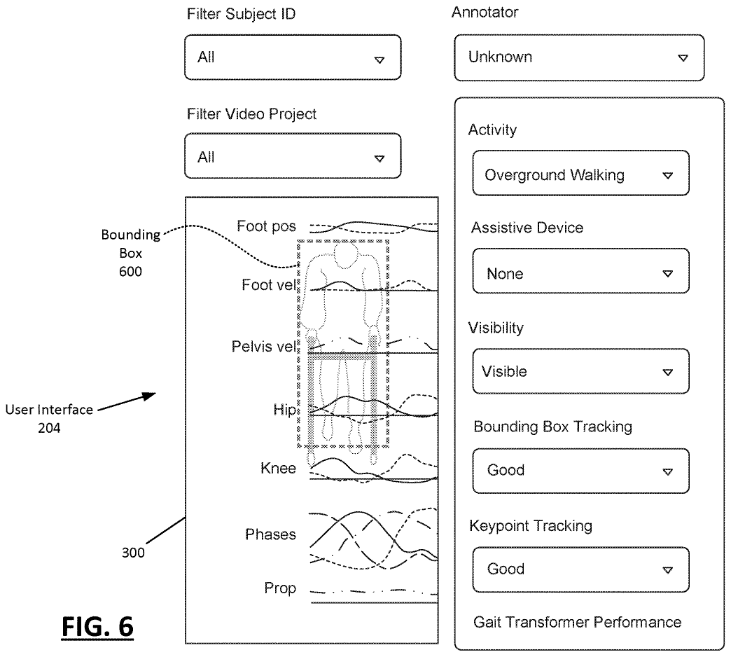

Portable Biomechanics Laboratory (PBL) dataset: Referring to FIGS. 4A through 4D, the PBL database is used such that another 4285 videos were collected in the clinic and 1938 videos were collected simultaneously with the MMMC pipeline 106. This data collection is similar to work validating the Gait Transformer 112 in clinical settings and includes additional wearable sensor measurements. All of the data collected from the PBL totals 78 hours. In addition to overground walking, it includes various activities performed during physical therapy, including walking outdoors, treadmill walking, stairs, running, backward walking, transitions from sitting to standing and back to sitting, and agility drills. To handle this data heterogeneity, an annotation tool was developed that extracts a random 10-second clip of video and allows for annotation with additional information about the activity, assistive devices, and performance of existing HPE algorithms (see FIG. 6).

FIG. 6 shows an example of an annotation tool provided via the user interface 204 that is used to label 10-second clips of video collected with the PBL. For example, a plurality of clips (e.g., 1800 clips) have been annotated with this tool. A video data 300 may be shown as a screen capture (static image) or as a video clip (moving image). The video that is annotated may include data such as feet position, feet velocity, pelvis velocity, hip positions, knee positions, and phases associated therewith, which may be superimposed on the video data 300. Additionally, the subject may be located using a bounding box 600 that tracks the location of the subject in the video. The user may then review the video and provide the necessary annotations (or make corrections to incorrect annotations), such as what the activity pertains to in the video, how the visibility of the subject is in the video, how well the bounding box is tracking the subject, how good the key point tracking in the video is, and/or how good the gait transformer performance is. Other fields may be included for providing improved training datasets.

Clinical Correlates: Clinical data associated with these datasets is also used for training the FMGA. This includes rehabilitation diagnosis, age, height, and sex. Longitudinal results from over 5000 clinical assessments performed during routine care, like the 10-meter walk test, the 6-minute walk test, the Berg balance scale, the Functional Gait assessment, and the lower extremity Fugl-Meyer assessment are also included. These datasets permit testing the ability of movement quality output to prognosticate future outcomes. This data is used to evaluate the performance of the FMGA on clinical tasks and to ensure that the model is generalizable to a wide range of clinical populations. This dataset also includes dictation of clinical impression of walking.

Gait laboratory dataset: This is a dataset of formal, clinical grade gait analysis results collected over a decade in a clinical gait laboratory. It contains data from 934 subjects during 1262 sessions: subjects' ages ranged from 2 to 87, and diagnoses include cerebral palsy and spina bifida, stroke, traumatic brain injury, spinal cord injury, amputation, and unspecified abnormality of gait. More than 9000 gait trials include a frontal plane video with synchronized motion capture data and force plate data, and an additional 3000 video-only trials have sagittal and frontal plane walking. Trials with motion capture data are fit with biomechanical models to get joint kinematics and kinetics (joint torques and ground reaction forces) from force plate measurements. This dataset also includes clinical assessments of spasticity, strength, and range of motion.

AMASS47 and BABEL48: In some examples, multiple motion capture datasets from a variety of sources are aggregated. Such aggregated datasets may be referred to as AMASS47 and BABEL48. The dataset contains data from 500 subjects performing nearly 18,000 motions. These motion sequences are all in a skinned multi-person linear (SMPL) format, which is a parametric mesh format. It has the benefit of being widely used, but the kinematic model used is not biomechanically ground. This dataset provides 3D joint locations, and by creating virtual camera models, these can be projected down to 2D joint locations. A range of activity labels are available depending on the specific datasets within this aggregate, and BABEL48 provides detailed granular action annotations.

### Methods of Using and Training the FMGA Model

The FMGA system 100 improves upon the Gait Transformer 112 and self-supervised learning pipeline 116 approach to gait-based biomarkers by training a transformer architecture on an extensive dataset of clinical movements. Like the SSL biomarker methodology, the FMGA system 100 uses masked sequence modeling in the pretraining stage, similar to pretraining Bidirectional encoder representations from transformers (BERT) language models.

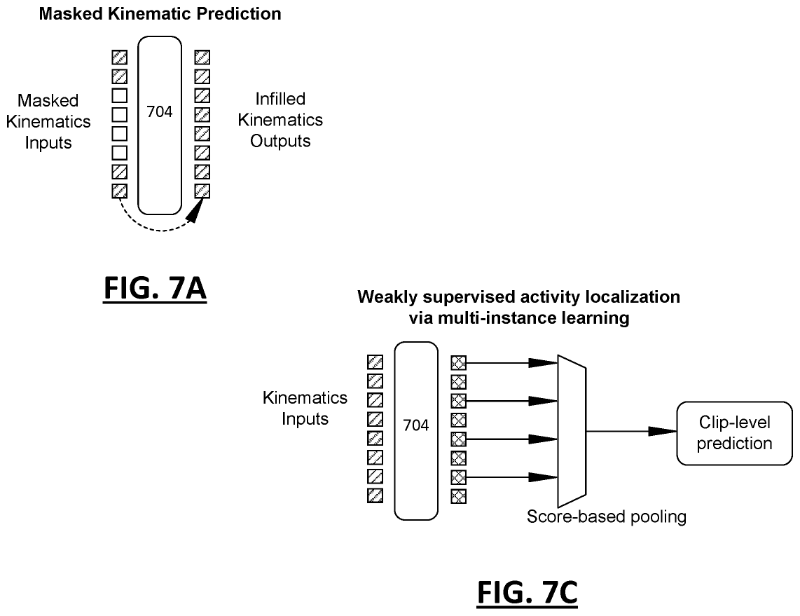

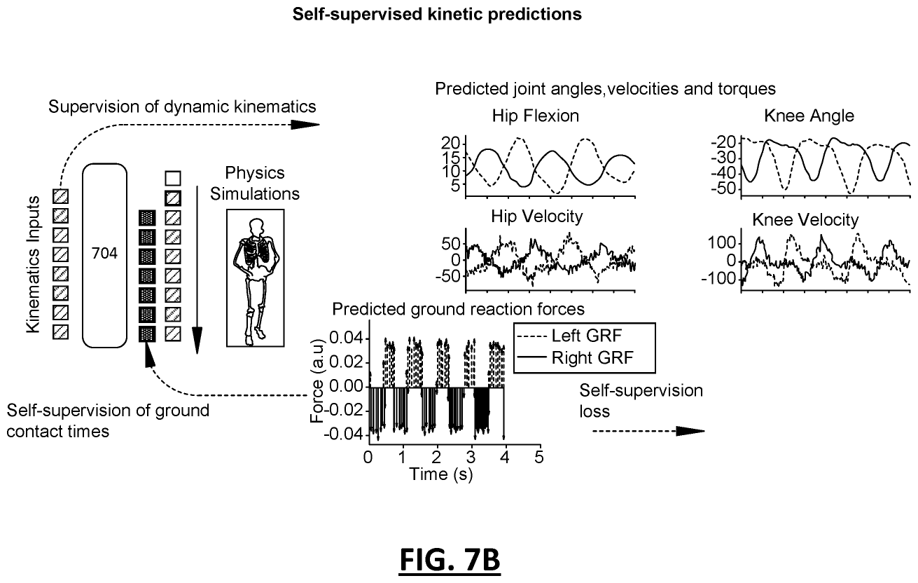

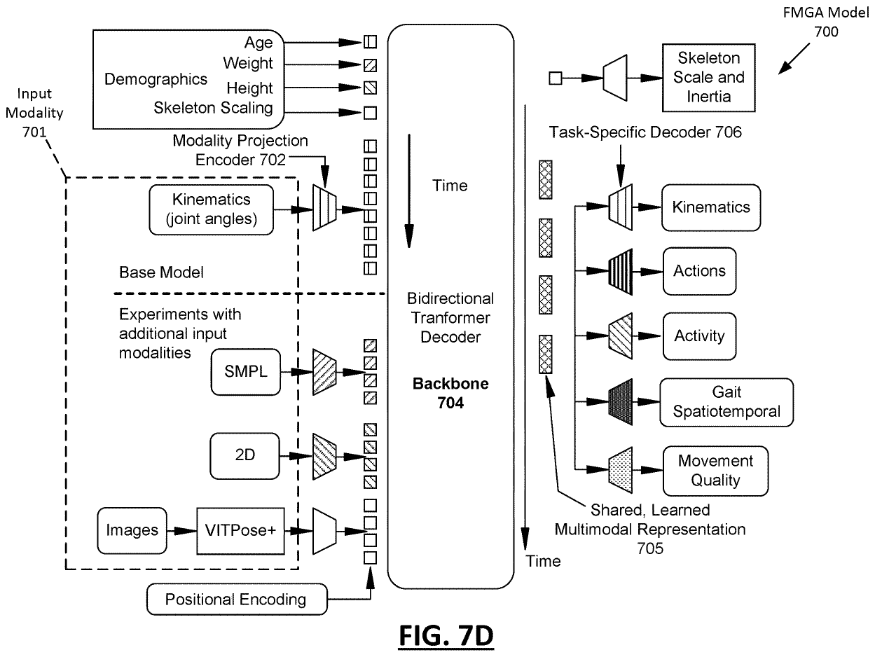

FIG. 7D shows an overview of a multimodal, multitask foundation model 700 as implemented in the FMGA system 100. In the model 700, each input modality 701 is passed through a separate projection head or encoder 702, with the sequence of these inputs processed by a shared encoder backbone 704. The backbone 704 outputs a shared, learned multimodal representation 705 that is then passed through a separate decoder head 706 for each task, such as producing joint forces or temporally localizing activities.

A modality projection (also referred to as the encoder 702) is a transformation operation that converts data from one modality-specific representation into another representational space. Modality projections are employed to align representations from different modalities so that they can be meaningfully combined or compared, which allows the model to perform cross-modal reasoning, such as answering questions about images or generating descriptions of visual content (e.g., questions regarding kinematics, actions, activity recognition, spatiotemporal gait parameters, and/or movement quality). In the context of FIG. 7D, the outputs from the modality projection 702 may include positional encoding.

A multimodal representation 705 is to a unified data structure that encodes information from multiple different input modalities into a common representational space. The multimodal representation enables a machine learning model to reason across these different types of inputs by mapping them into a shared embedding space where relationships between concepts across modalities can be learned and exploited. The multimodal representation 705 can be decoded into individual tasks using the task-specific decoder head 706 such that tasks associated with, for example, kinematics, actions, activity recognition, spatiotemporal gait parameters, and movement quality can be decoded from the multimodal representation 705 using the corresponding decoder head 706.

As shown in FIG. 7D, the FMGA model 700 takes in a sequence of poses. These poses are projected into tokens passed through several layers of a bidirectional transformer (such as the backbone 704) to produce a series of output tokens. Separate heads decode these output tokens into final, interpretable outputs: joint angles, joint velocities, actions with the corresponding torques and ground reaction forces, fine-grained and localized activities being performed, and temporal localization. The transformer also outputs a token for movement quality, following the SSL biomarker methodology.

The FMGA model 700 is configured to handle the diverse body shapes and demographic ranges presented in clinical practice. Accurate anthropomorphic measurements, such as having the correct limb segment lengths when modeling kinematics, are essential in biomechanics. When modeling dynamics, it is also important to have accurate segment masses and moments of inertia. These are typically obtained for body segments through standard regression equations obtained from cadaveric and imaging studies. The FMGA system 100 takes in standard demographic information (e.g., age, height, and weight) and the limb segment lengths obtained from kinematic fits and outputs the scaling parameters and inertia matrix required for dynamics. These outputs are penalized by deviations from the standard regressions so as to increase the accuracy of the outputs. Where the FMGA system 100 is driven directly from images, these parameters may be directly inferred.

FIGS. 7A through 7C are visualization of some of the key losses that will be combined for training the model using the backbone 704 of FIG. 7D. For example, FIG. 7A shows a masked prediction of the kinematic trajectories, where the self-supervised loss is from the masked kinematics inputs to the infilled kinematics outputs. FIG. 7B shows self-supervised kinetic predictions where the predicted actions will be fed into dynamic predictions to produce torques and ground reaction forces. These will be self-supervised to match the input kinematics and will supervise the ground contact outputs. For example, the predicted GRFs (which includes left GRF and right GRF as shown) can be used for self-supervision of ground contact times, and the dynamic kinematics can be used for supervising predicted joint angles, velocities, and torques (such as hip flexion and hip velocity, as well as knee angle and knee velocity, for each of left and right sides of the body, as shown). FIG. 7C shows a weakly supervised activity location via multi-instance learning via score-based pooling for clip-level prediction, where the localized class activation sequence is pooled for clip-level activity prediction that supervises the fine-grained predictions.

Training for dynamics—torques and GRFs: To compute actions and the corresponding torques and GRFs, the FMGA system 100 utilizes the Decision Transformer and Masked Trajectory Modeling. These methods transform reinforcement learning problems into sequence modeling problems, where the sequence includes mixtures of actions and states. After masked sequence pretraining, these models can solve inverse dynamics problems like computing a sequence of actions (torques) from a sequence of observed states (kinematics). Having the transformer tokens outputs for each time jointly represent both states (joint angles and velocities) and actions has been shown to outperform prior works treating these as separate tokens.

Notably, these methods use heteromodal data that combines data with ground truth actions and those without. Through training, the transformer internalizes the system dynamics (world model) of the system being modeled. However, much of the data used for training the FMGA system 100 is acquired with video-only methods that lack any measurements of GRFs. Thus, the FMGA system 100 utilizes an approach to learn actions without ground truth measurements. The physics simulator (e.g., MuJoCo, MyoSkeleton), and the biomechanical model provide the world model to make this feasible. During training, the implicit kinetic method is copied using the predicted actions to produce short-horizon dynamic simulations. These are supervised to match the input trajectory. By backpropagating through the transformer, the FMGA system 100 learns to predict consistent state and action trajectories guided by the input set of unmasked state observations (FIG. 7B). These dynamic rollouts 212 that are trained to match the observed kinematics are similar to the implicit kinetic approach described above (such as in FIGS. 2 and 4).

Training the FMGA system 100 on this task accelerates inference compared to the existing end-to-end optimization for each trial, which is of practical importance for clinical deployment. Furthermore, the FMGA system 100 produces a deeper understanding of human movement in the transformer, thereby resulting in positive transfer learning for other tasks. The FMGA system 100 is also trained on data from a clinical gait laboratory dataset, which includes ground-truth ground reaction force measurements, which is also used to validate the approach.

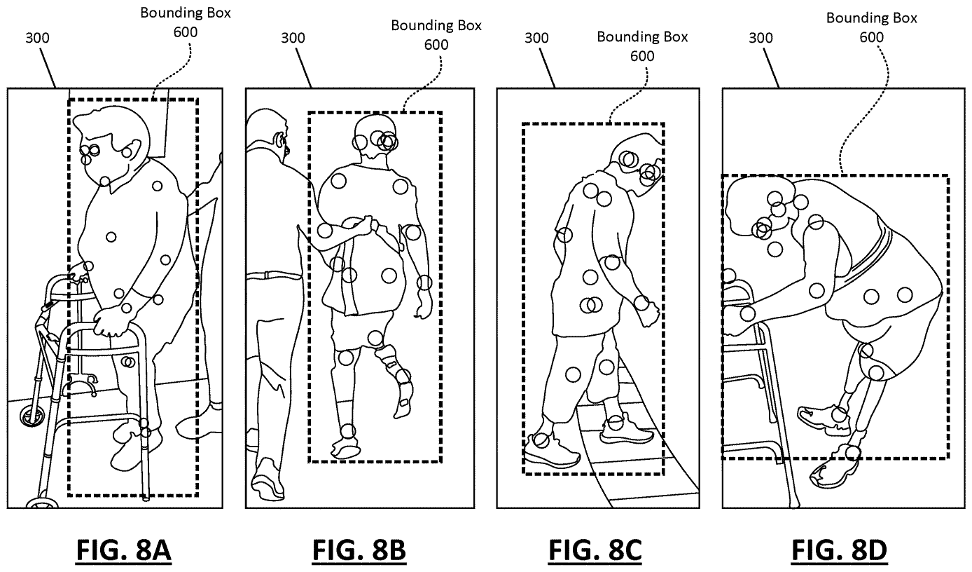

Training for activity recognition and localization. Clinical data subjects perform a wide range of activities that are either not represented in the annotations of typical action recognition algorithms or are misidentified when performed by an individual with impairments. In each of FIGS. 8A through 8D, the video data 300 is provided showing a person in certain types of motion, and the bounding box 600 is superimposed to track the location of the subject in the video data 300.

In FIG. 8A, the typical action recognition algorithm may recognize the person who is walking slowly using a walker to be classified as standing even though the leg is in mid-swing position. In FIG. 8B, the algorithm may recognize the person running with a carbon fiber prosthesis to be classified as standing. In FIG. 8C, the algorithm may recognize the person using a ladder for agility training to be classified as standing. In FIG. 8D, the algorithm may recognize the person using bilateral lower-limb prosthesis with a rolling walker to be classified as taking a bow when the person is actually sitting. The FMGA system 100 is trained on a database of movements containing a plurality of annotations of clinical activities, such as walking, walking in tandem gait, walking backward, performing a Timed-up-and-Go (TUG), Four-square-step-test (FSST), Functional Gait Assessment (FGA), and numerous upper extremity assessments. These annotations are available for clips collected with the Portable Biomechanics System deployed in a gait clinic that were created with an annotation dashboard (see for example FIG. 6), as well as from the MMMC pipeline 106. These annotations are harmonized into a set of clinically relevant activities.

These clip-level annotations, however, do not capture the exact timing of different activities. Knowing the activity and when it was performed is essential for successful clinical deployment. Real world videos are often long and unstructured, but it is desirable to extract specific activities for more precise analysis. For example, it is irrelevant to analyze gait spatiotemporal parameters if the subject is not walking. Localizing actions from clip-level annotations is referred to as weakly supervised activity localization. Losses and methods for this task are incorporated in training the FMGA system 100. Specifically, FMGA training methodology builds upon approaches which perform weakly supervised activity localization for skeleton representations to obtain frame-level activity labels. At a high level, this uses multiple instance learning (MIL), which treats the individual frame predictions as aggregated instances. This aggregation predicts class activation scores for each frame based on cosine similarities between the extracted features and predefined weight vectors for each action category. These frame-level predictions are treated as pseudo-labels and refined iteratively through joint optimization. This joint optimization involves alternately updating the network parameters and the pseudo-labels. During this process, noisy pseudo-labels are gradually corrected as the network's predictions become more accurate. A temporal contrastive loss on the activity head, where temporally close samples are pushed toward each other, may also be included.

Movement quality and gait-based biomarker. Contrastive loss applied to an additional movement quality token from the SSL biomarker methodology may also be included. This takes different snippets from trials as positive examples and pushes the representation closer together while pushing it away from any other trials. This is only applied to walking trials, as the idea that different times in a trial are roughly the same is more appropriate for stable walking than multi-step tasks like a TUG. Additional linear decoders are trained on this representation to decode clinical annotations, including decoding the diagnosis and testing how well the system can predict the performance on different outcome measures from the kinematics of walking alone.

### Metrics

Torques and ground reaction forces. Accuracy of estimated ground reaction forces is compared to the ground truth measured in the clinical gait laboratory dataset. This is computed as the root-mean-squared error (RMSE) in both Newtons and as a percentage of body weight. Torques from the clinical gait laboratory dataset will also be used for validation. Data used for validation is excluded from training the FMGA system 100.

Gait event timing. Accuracy of gait event times is determined by thresholding the ground reaction forces. Trials obtained with paired recordings from an instrumented walkway are used. This walkway also reports the location of the center of pressure for each foot as a function of time, which is used to compare against dynamic physics simulator rollouts. The FMGA system 100 is expected to be more accurate than the Gait Transformer, which has a median error of 25 ms.

Activity recognition and localization. Clip-level activity recognition accuracy is measured on a validation dataset and the timing error. This is achieved with manual annotation of the beginning and ending of 250 trials of movements from multicamera and single-camera systems. Timing error of the activity boundaries are reported.

SSL Biomarker. The contrastively learned token from the transformer was found to contain rich health information that could be decoded. A decoder is built for diagnosis and gait impairment laterality to measure the accuracy of this biomarker.

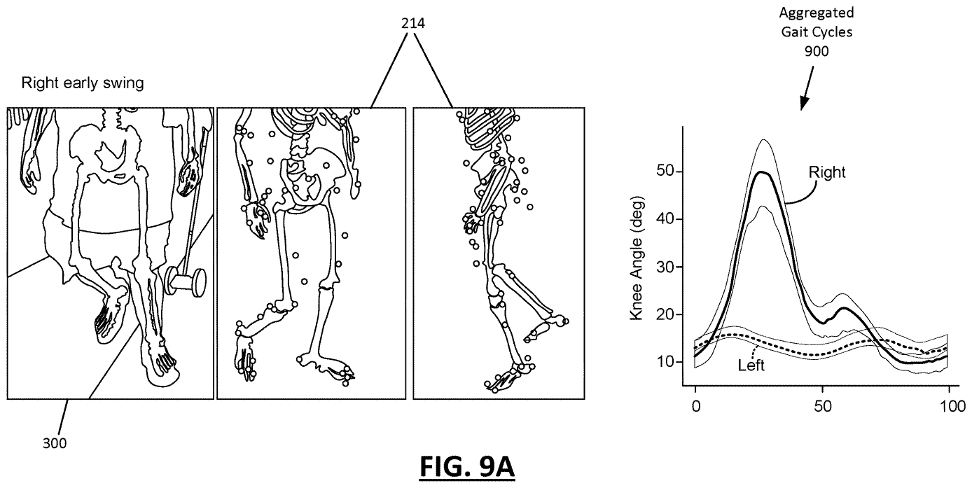

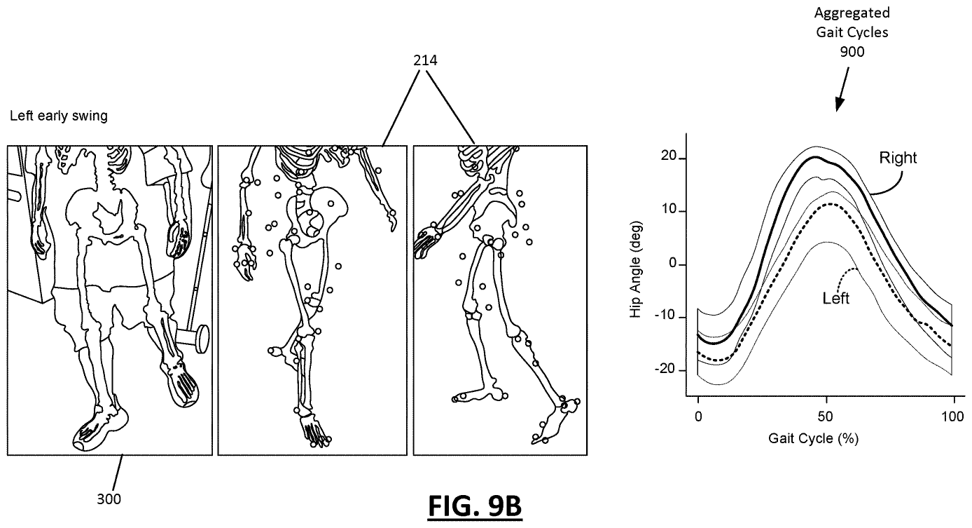

Gait Analytics Dashboard. The FMGA model 700 can reliably localize walking within clips, identify gait cycles from when the ground reaction force indicates foot contact, and measure kinematics and kinetics in that time window. A dashboard to visualize these results is also provided herein. FIGS. 9A and 9B show exemplary waveforms using existing kinematic reconstruction with the gait cycles extracted from the Gait Transformer 112 based on the manual annotation of walking trial. The participant shown in FIGS. 9A and 9B has left hemiplegia, so there is reduced knee flexion on paretic side during early swing. Also shown are aggregated gait cycles 900 for the knee and hip, quantifying this impairment. A dashboard is provided to visualize these results from the FMGA system 100, including the kinetic results and averaged ground reaction force. The dashboard may include the video data 300 and the virtual skeletal reconstruction 214 based on the video data. The video data 300 may also include the virtual skeletal reconstruction 214 superimposed thereon to show how the model interprets the skeletal form of the subject in the video data 300.

Test for Positive Transfer Between Tasks. A motivation for developing the FMGA system 100 instead of training a separate network is that it will provide positive transfer learning between tasks, where the performance of individual tasks improves with the network being trained on additional tasks.

### Alternative Approaches

The physics simulator may be run at 30 Hz frame rate when training dynamics outputs. Alternatively, a given action may be run for N repetitions to obtain stable performance. Higher frequency tokens may be masked and the FMGA system 100 may be trained to infer faster dynamics and states than the input framerate. The FMGA system 100 may be trained to produce inferences at a higher rate than the sampling rate or to consume variable rate videos such as 60 Hz and 120 Hz smartphone videos.

The duration of physics rollouts may also be tuned. For example, rollouts of several frames may provide stronger dynamics supervision. However, because the predicted actions are run without feedback (in contrast to a reinforcement learning policy that only predicts a single step), rollouts too long may cause excessive tracking errors that harm training. Regularization may therefore be added to encourage the predicted actions to be smooth, which may be implemented through an energy regularizer.

In one example, a simple bidirectional multi-head transformer is used to produce the sequence of tokens representing poses and demographic information. Alternatively, a dual-stream spatiotemporal transformer may be used.

Additional unsupervised learning losses for hierarchical activity recognitions may also be implemented. For example, reusable atomic actions (e.g., sit, stand, turn) that can occur in certain clinical activities (e.g., both TUG and the L-Test incorporate sit, stand, and turns, but in a different sequence) may be incorporated.

### Additional Experiments

Directly incorporating vision, key points, and other body models. For input into the FMGA system 100, kinematics may be computed from either monocular or multi-view data, where key points are extracted from images. Alternatively, the FMGA system 100 may be configured to take in key points directly or learn features extracted from images. Particularly in the case of monocular video, driving with images or key points may improve performance if there are viewpoint-dependent biases in the 3D key points that are used in kinematic fitting. For directly driving from vision, features from VITPose+, which uses a vision transformer that incorporates a task-specific mixture of elements in the feedforward layer to improve the handling of different key point sets, will be used. Using vision inputs may permit the system to directly infer the skeleton scaling by masking these tokens during training while still providing the demographic inputs.

Pretraining on public datasets. The FMGA system 100 may also be trained on existing public movement datasets. The largest public dataset, AMASS, uses the SMPL model. This is very popular in the computer vision community and is optimized to capture the contours of body models, but it uses a non-physiological kinematic tree. To bridge this gap, the system may include an additional input modality and projection layer that accepts SMPL body parameters. An additional loss may also be included that supervises the output biomechanical parameters to produce consistent joint locations as in the SMPL model. This is facilitated by recent work linking the SMPL models to a biomechanical cousin, SKEL. With this, the FMGA system 100 may serve as a Rosetta stone to translate from this popular, non-physiologic representation into a proper biomechanical format. Furthermore, the AMASS dataset has been annotated with fine, granular activity labels, which can be used to train activity localization outputs. While this dataset does not include many participants with movement impairments or clinical assessments, including this large dataset may produce positive transfer learning for clinical tasks.

### Natural Language Multimodal Interface

Also presented herein is a system comprising a trained language interface that can process human movements and language, such that the trained language interface has been validated to answer clinically relevant questions with accurate information. The FMGA system 100 may be integrated with a natural language multimodal interface trained to interact with movement data and text, to solve clinically relevant open-ended tasks. For example, the natural language interface (e.g., the user interface 204) is configured to reliably provide accurate answers to a range of questions helpful to clinical users when given a mixture of language and motion tokens.

The inventive system develops a motion vocabulary, or tokenizer, and then trains a language model to take in these motion tokens in addition to language and to fluently respond with both. Essential differences between the present system and prior work are 1) it uses movement defined on a biomechanical model instead of non-physiological models popular in computer graphics, 2) it is trained on data that includes movement impairments, and 3) the prompts it is trained on reflect questions and answers helpful to a clinician.

Training data includes over 100 hours of movement data, mainly from clinical populations, acquired from either the MMMC pipeline 106 (45 hours) or the proprietary smartphone system (78 hours, 10 of which overlap with MMMC). Additionally, this training data has a rich set of annotations for building the clinically oriented paired language-motion dataset. This includes: (a) basic demographic information (age, sex, height, weight) and rehabilitation diagnoses (stroke, spinal cord injury, traumatic brain injury, lower-limb prosthesis user, neuropathy, cerebral palsy), (b) manual annotation of the activities from over 9000 clips, ranging from walking to components of clinical outcome assessments, and (c) from over 500 of the participants, the results of thousands of standard clinical outcome assessments longitudinally over time. A dashboard for clinicians to dictate their impressions from videos is also used to collect training data.

Methodologies for linking language to motion modeling typically focus on generating movements from a motion prompt, often using language-conditioned diffusion. While this may have some didactic and research utility, it is a less helpful task in clinical practice. Another approach combines still images, single SMPL poses, and language as inputs and produces an SMPL or language output. However, most of the clinically pertinent aspects of movements are in the temporal dynamics, and processing only still images or poses is overly constraining. Furthermore, these approaches do not leverage the full power of modern large language models (LLMs).

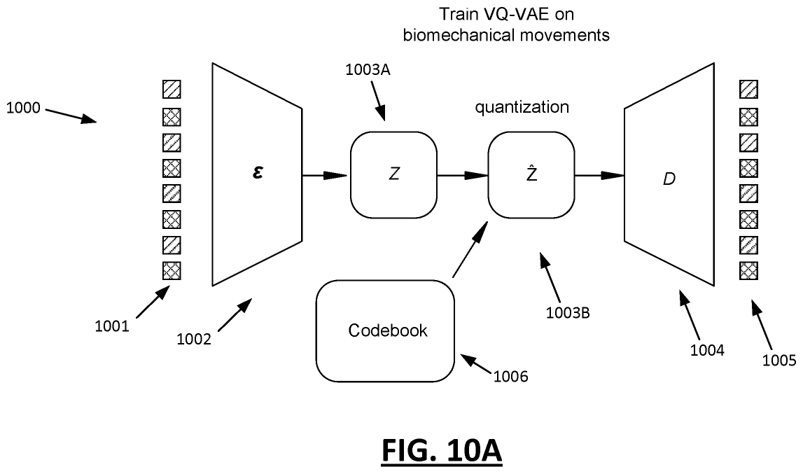

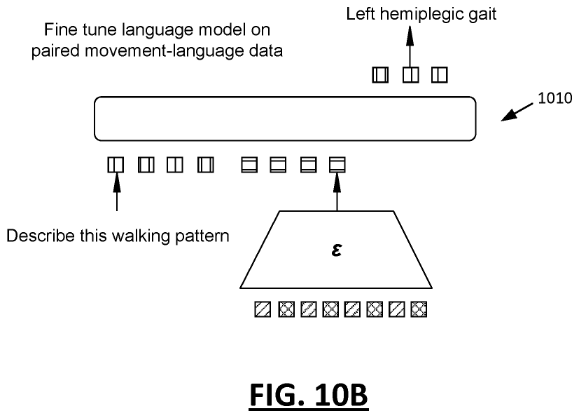

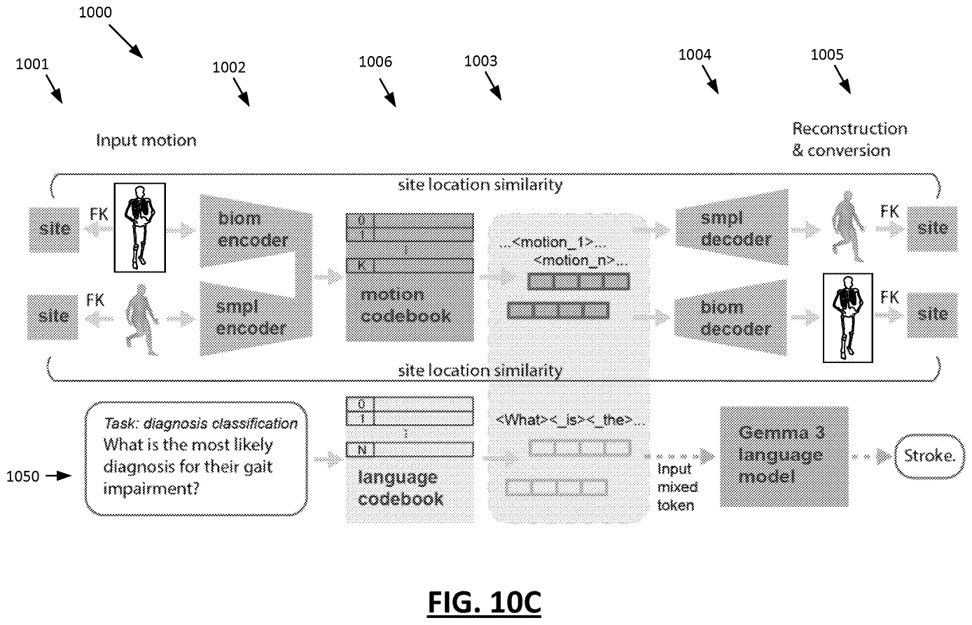

The present system provides a clinically useful natural language interface that takes in biomechanically grounded trajectories as is validated for people with movement impairments (FIGS. 10A and 10B). In one aspect, the system comprises a model that treats movement the same way large language models treat text—as a sequence of tokens that can be encoded, generated, translated, and reasoned about using language-model techniques. The system tokenizes movement as a foreign language with a vector-quantized variational autoencoder (VQ-VAE), which is a type of generative model that combines aspects of variational autoencoders with discrete latent representations. Unlike traditional variational autoencoders that use continuous latent spaces, a VQ-VAE employs a discrete codebook of embedding vectors to represent encoded data.

In operation, referring to FIG. 10A, a VQ-VAE model 1000 includes an encoder 1002 that maps input data 1001 (e.g., video data, sensor data, and/or data from biomechanical models, or kinematic movement data derived from such data) to a continuous latent representation (at representation block 1003A). This continuous representation is then quantized (at quantization block 1003B) by finding the nearest embedding vector in a learned codebook 1006. The block 1003 is thus associated with a latent space. The quantized representation is subsequently passed to a decoder 1004 that reconstructs the original input from the discrete latent codes as output 1005. Specifically, FIG. 10A shows the training process for a VQ-VAE model 1000 to tokenize movement sequences, where biomechanical movement data 1001 is encoded using an encoder 1002, then vector-quantized using vector-quantization (block 1003), after which it is decoded using a decoder 1004 to generate outputs 1005. A codebook 1006 is used as part of the tokenization process such that the encoder 1002 embeds an SMPL sequence into a latent token using the learned codebook 1006. FIG. 10B shows another training process for a large language model 1010 on paired language and movement. The LLM 1010 is fine-tuned with a dataset of paired motion and language.

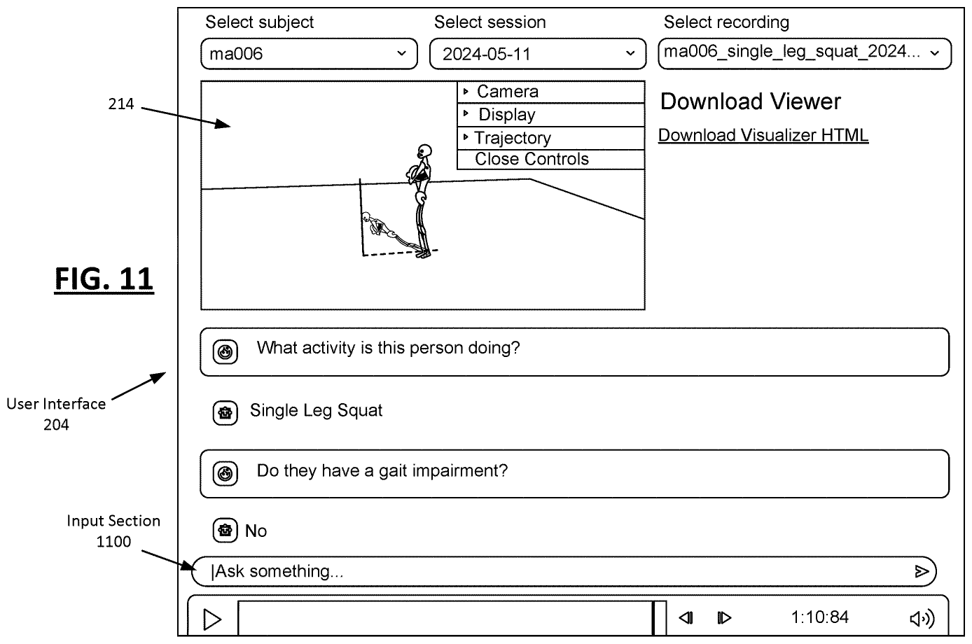

FIG. 11 shows a user interface 204 used in a biomechanics LLM. There is an input section 1100 to ask the model to respond to basic questions, such as “What activity is this person doing?” or “Do they have a gait impairment?” based on the virtual skeletal reconstruction 214. The biomechanics LLM addresses the challenge of data collection being easier whereas the analysis is more difficult. The purpose of the biomechanics LLM is to train a multi-modal language model to be fluent in the movement of a human being. To do so, movements are tokenized as a foreign language with the VQ-VAE model as explained above.

Several performance metrics may be derived from the confusion matrix. Accuracy is calculated as the ratio of correct predictions to total predictions. Precision is calculated as the ratio of true positives to the sum of true positives and false positives. Recall, also known as sensitivity, is calculated as the ratio of true positives to the sum of true positives and false negatives. F1 score represents the harmonic mean of precision and recall. In the context of LLMs, confusion matrices enable identification of specific classes that the model frequently confuses, thereby facilitating targeted improvements to model training or architecture. Such LLMs may be used in tasks including but not limited to: activity classification, task segmentation, scoring clinical outcome assessments (COAs), and gait analysis.

Agentic Architecture and Benchmarking: In some examples, the biomechanics LLM of FIG. 11 incorporates a code-generating artificial intelligence (AI) agent that enables biomechanical analysis through natural language and allows users to querying databases, generating visualizations, and interpret data without requiring users to write code. To evaluate the capabilities of this AI agent, a systematic benchmark spanning data retrieval, visualization, activity classification, temporal segmentation, and clinical reasoning may be applied. It has been shown that the AI agent achieves robust accuracy on data retrieval and visualization tasks and demonstrated emerging clinical reasoning capabilities. Biomechanically-informed, domain-specific instructions significantly improve performance over generic prompts, and integrating validated specialized tools for gait event detection substantially boost accuracy on challenging spatiotemporal analysis where the base agent struggled.

As further explained herein, a multimodal LLM that is fluent in both biomechanics and language may be implemented in association with the code-generating AI agent. Multimodal LLM is trained to answer clinically relevant tasks in several domains; for example, multimodal LLM shows high performance at classifying activities or detecting impaired gait. However, in some examples, multimodal LLM can only answer the classes of questions for which it is trained and/or may make errors (hallucinations) without the capability to explain the rationale for its answers. To remedy such shortcomings that are experienced by many LLMs, the multimodal LLM as presented herein are systematically benchmarked, and its performance is measured across multiple domains to better understand its performance limits, thereby guiding future improvements and inform end users of the tasks for which the multimodal LLM can be trusted.

However, one of the challenges for benchmarking agentic systems is that, while the potential uses of the code-generating AI agent are open-ended, quantitative and interpretable benchmarking requires defining a discrete set of tasks. To address this, in some examples, the benchmarking process begins with open-ended user testing to identify some example use cases. From these, several domains of tasks, including targeted data retrieval, data visualization, activity classification and segmentation, and even clinical reasoning, may be established. In some examples, additional ground-truth data that was used to develop the benchmarking dataset, including clinical reasoning and activity classification, may be tested in a two-alternative forced-choice (2AFC) paradigm. In some cases where it is harder to define an exact answer, an LLM-as-a-Judge framework may be implemented to evaluate the performance of the code-generating AI agent.

According to embodiments disclosed herein, the AI agent associated with the biomechanics LLM of FIG. 11 is a code-generating AI agent architecture built on a “smolagents” framework (which is an open-source Python library designed to simplify the process of build and run agents using fewer lines of code). Unlike function-calling approaches, in which language models output structured JSON to invoke predefined functions, smolagents generates and executes Python code directly, which is effective for expressing agentic plans. The AI agent receives a natural-language query (for example, via the user interface 204), reasons about which computational steps are required, writes executable code, observes the results in a sandboxed environment, and iterates until a final answer is reached.

According to examples, the AI agent accesses specialized capabilities through special Python functions called “tools.” These tools provide database access for querying participant trials and fetching kinematic data, gait analysis for detecting foot contact events from kinematics, and visualization for generating static and interactive plots. Tool documentation embedded in function definitions informs the AI agent how to use each tool, including the expected inputs and outputs, and the AI agent then writes code to flexibly use these tools to retrieve and analyze data.

Upon receiving the user prompt, the AI agent begins emitting text, including flags indicating that sections are code. The smolagent framework extracts those from the LLM as they are produced, and when a complete block of code is received, the block of code is executed and used as the new input during a chat sequence. Once the AI agent determines that the question can be answered, the AI agent responds with a “final_answer” tool call which stops the sequence.

Language Model Backends: The AI agent may be evaluated with language-model backends, representing cloud and local deployment scenarios. For example, models capable of processing images and text may be used, including but not limited to Gemini 2.5 Flash Lite (a frontier cloud-based model) and MedGemma 27B (an open-weights medical foundation model), both of which can process images and text. Whenever the AI agent generates an image and uses the “save_plot” tool, the image may be presented to the end user and provided to the visual language model (VLM), along with the text, for subsequent chat iterations.

Custom Instructions: Domain-specific guidance may be embedded in the AI agent's system prompt to prevent common errors discovered during early testing. Such instructions may include biomechanical formulas (e.g., the calculation of the asymmetry index) and/or expected parameter ranges, and may also provide guidance on standards, such as using consistent colors to indicate left and right, using real units of time and degrees on the axes, and/or avoiding common errors (e.g., forgetting to import mathematical libraries or plotting tools). Domain-specific guidance may encourage the AI agent to reason about clinical questions and to formulate hypotheses to explore in the data.

Data Sources and Access Privileges: The AI agent may use data sources such as kinematics data source, including but not limited to multi-view marker-less motion capture devices. Data may be managed via a suitable processing framework, such as DataJoint and the PosePipe processing framework. However, in some examples, separation exist between data sources available to the AI agent and those used solely for ground-truthing during dataset generation. For example, during evaluation, the AI agent may have access only to kinematic reconstructions and activity annotations derived from video, but does not have access to: instrumented walkway (per-step spatiotemporal measurements such as velocity, step length, and/or stride time, that serve as ground truth for gait parameter estimation); clinical records (diagnosis labels such as stroke, prosthesis user, and control, as well as affected side and/or level of amputation); Berg Balance Scale assessments (standardized fall risk scores obtained within a week of a gait trial for inpatients); and/or activity segmentation annotation (manual labels of activity segmentation, such as rising from a chair, turning, or the push phase of wheelchair propulsion).

Chat Interface: As shown in FIG. 11, the end-user chat interface (e.g., user interface 204) may be implemented using any suitable data application framework, such as “streamlit” which is an open-source Python framework that is simplified for data scientists and artificial intelligence/machine learning engineers to deliver interactive data apps using fewer lines of code. The user inputs are provided to the AI agent via the input section 1100 of the chat interface, and the resulting code and plots are shown to the user as they are generated until the AI agent emits a “final_answer” tool in response to the input. At that point, the user can either start a new chat or ask a follow-up question to continue the existing chat interface.

Types of Tasks: Numerous types of tasks may be provided for the AI agent: (1) database tasks, (2) activity classification tasks, (3) spatiotemporal tasks, (4) clinical tasks, (5) segmentation tasks, (6) visualization tasks, and/or (7) perception tasks. Details regarding each task are explained herein.

Database tasks test the AI agent's ability to query and aggregate information from the motion capture database. For example, questions may span trial counts (“How many trials does participant X have?”), data shapes (“What are the dimensions of the kinematics array?”), activity queries (“List all TUG trials for participant X”), session information, and/or date spans. Ground truth is computed via direct queries to the database using validated calls. These tasks are deterministic and verify that the agent correctly interfaces with the database.

Activity classification tasks present two-alternative forced choice (2AFC) questions requiring the agent to identify which of two trials contains a specific activity by analyzing kinematics alone. Such questions may include, for example, backward walking versus forward walking, Timed Up and Go versus overground walking, etc. Success requires identifying discriminative kinematic features, such as: reversed ankle plantarflexion-dorsiflexion sequences for backward walking, characteristic turning patterns for TUG, and/or altered coordination patterns for different gait conditions. The AI agent may be specifically instructed not to use activity annotations, and the tool providing activity labels may be blocked via a forbidden tools constraint to facilitate enforcement thereof.

Spatiotemporal tasks evaluate estimation of gait parameters from kinematics without access to instrumented walkway measurements. Topics may include, for example: velocity and spatial parameters (walking speed, step length, stride length), temporal parameters (stride time, stance and swing duration, stance/swing ratios), gait event detection (identifying stance and swing phase timing), event counting (number of left and right foot contacts), and/or asymmetry metrics (left-right differences in step length and timing).

Clinical tasks require inferring clinical status from kinematic patterns. For example, in a 2AFC format, the AI agent may be asked to: determine whether one of two participants has a stroke (e.g., versus an able-bodied control), determine whether a participant uses a lower-limb prosthesis, identify the affected side, and/or compare fall risk of the participant. Ground truth may come from clinical records and Berg Balance Scale assessments. In some examples, success of the AI agent requires recognizing clinically relevant patterns, such as an asymmetric range of motion.

Segmentation tasks test the identification of temporal phases within complex activities. Questions may address phase duration (“How long was the turning phase?”), timing (“When did the sit-to-stand transition begin?”), and/or counting (“How many walking segments occurred?”). Ground truth may come from manual video annotations. These tasks evaluate whether the agent can parse continuous motion into clinically meaningful segments.

Visualization tasks assess the AI agent's ability to generate appropriate plots for different analytical needs. Examples of such analytical needs may include: single joint trajectories, bilateral comparisons, multi-joint summaries, pelvis trajectories, and/or range of motion summaries. In some examples, the evaluation uses an LLM-as-judge approach with domain-specific rubrics to assess whether the visualization addresses the request and displays appropriate data.

Perception tasks evaluate the agent's ability to interpret time series plots and extract meaningful observations. Unlike visualization tasks which test plot generation, perception tasks may require the agent to first create a visualization and then accurately describe what it observes. Topics may include, for example: clinical pattern recognition (detecting asymmetry in range of motion that is indicative of stroke or prosthesis use), activity pattern recognition (identifying turning, backwards walking, or head nodding from kinematic traces), movement phase identification (recognizing sit-to-stand transitions, walking onset, or L-shaped trajectories), and/or gait cycle analysis (estimating cadence, assessing stride symmetry). In some examples, the key evaluation criterion is whether the AI agent's description is concordant with the expected pattern given ground truth clinical or activity information that the agent cannot access directly. LLM-as-judge scoring emphasizes specific value citations over generic descriptions.

Agentic Scoring: Deterministic scoring may be applied where ground truth permits objective evaluation. Categorical responses (activity names, laterality, and/or participant identifications) require exact string matches. Numerical responses are specified with tolerances appropriate to measurement uncertainty (e.g., ±10% relative tolerance for most spatiotemporal parameters such as velocity, step length, and/or temporal measures, ±5 percentage points for asymmetry metrics, and/or ±1 for footfall counts). In some examples, exact matches may be applied for database counts and data shapes.

LLM-as-judge may be used for tasks requiring semantic evaluation, particularly visualization quality, and clinical reasoning, for example. Domain-specific rubrics score multiple dimensions on a 0-10 scale. Importantly, the rubrics evaluate reasoning quality alongside correctness. For example, well-reasoned answers that reach incorrect conclusions would score higher than lucky guesses without justification, which encourages genuine analytical capability over pattern matching.

In some examples, the user interface 204 of FIG. 11 may include a Streamlit GUI to visualize agent behavior from the evaluation dataset. An example of such GUI may include a chat interface showing responses from the AI agent, including the reasoning traces, the code that was executed, and/or the plots that were generated by the AI agent. Other trackable metrics include token usage by the AI agent, execution time by the AI agent to characterize computational costs across configurations, and/or the number of coding errors produced by the AI agent.

The AI agent may be implemented in different configurations, including but not limited to: baseline (e.g., image-and-text processing model such as Gemini 2.5 Flash Lite, with custom biomechanics instructions), gait transformer (baseline with access to learned gait event detection tool), no instructions (with default prompts only, removing domain-specific guidance such as lesion study), no vision (baseline without feeding generated plots back to the model), and local (e.g., a different image-and-text processing model such as MedGemma Local 27B model for on-premises deployment)

No Vision Performance: Beneficially, it was discovered during performance evaluation of the AI agent as disclosed herein that the performance of the AI agent was high (e.g., greater than 90% accuracy) for visual estimation such as range of motion and gait event timing, regardless of whether the AI agent had access to the visual input data. Specifically, upon disabling the transfer of generated plots to Gemini's visual inputs and removing instructions that would otherwise prompt it to attend to plots, performance on most tasks was comparable or even better without visualization. This included tasks that were anticipated to benefit from graphing, such as clinical reasoning. For example, with regard to perception tasks that require interpreting generated plots (e.g., “describe the movement pattern you observe”, “estimate cadence from hip flexion peaks”) and visualization tasks that only require creating a correct plot (e.g., “plot the knee angle during walking”), vision capabilities helped with perception tasks, while visualization performance remained high regardless of vision. However, the model without vision was still able to write code that could solve many perception tasks, such as computing statistics or detecting peak locations in waveforms.

In summary, the AI agent as disclosed herein remains to be a very powerful and convenient tool for accessing and visualizing data. The consistent and robust performance of the AI agent in both data retrieval and plotting addresses a common request from clinicians using the marker-less motion capture system. The ability to ask in natural language (such as “please pull up the knee kinematics during a single leg squat for participant ABC and compare the first session to the last session”) lowers the barrier to working with biomechanical data. This complements the benefit of marker-less motion capture by lowering the barrier to data collection.

### Paired Language-Movement Dataset

Referring back to FIG. 10A, in some examples, the system presented herein includes a VQ-VAE model 1000 that is trained on human movement that generalizes to impaired movements. The LLM 1010 of FIG. 10B is fine-tuned with a dataset of paired motion and language including: (a) data subjects' demographic information, primary rehabilitation diagnosis, and comorbidities; (b) annotations for activities, assistive devices being used, and movement scores, such as the time for a timed-up-and-go, associated with data subject movement videos; (c) data subjects' walking features, such as spatiotemporal gait parameters, joint range of motion, and movement symmetry; (d) data subjects' performance on several other clinical outcome assessments performed as part of routine care; and (e) input from an annotation dashboard such as the user interface 204 similar to FIG. 6 that allows clinicians to speak a clinical impression of movement that is transcribed and stored in the database 104. Finally, because a plurality of data subjects are assessed at different times, prompts may be created that train the LLM to compare different walking patterns.

These annotations and clinical impressions are used to programmatically generate a large corpus of clinically-relevant sentences. For example, sentences such as:
QA: ‘What is a likely diagnosis for someone walking like this? <MOTIONSEQUENCE> This walking pattern is consistent with someone with a stroke influencing the left side of their body.’
QA: ‘What is this person doing? <MOTIONSEQUENCE> They are performed a timed-up-and-go.’
QA: ‘How well are they performing this task? <MOTIONSEQUENCE> This person performs a timed-up-and-go in 25 seconds, which is slower than the typical speed.’
QA: ‘What is the step length of this walking pattern? <MOTIONSEQUENCE> The left leg has a step length of 0.8 on the right and 0.3 on the left, with an asymmetry index of 0.27.’
QA: ‘What is the maximum knee flexion on the hemiparetic side? <MOTIONSEQUENCE> The maximum knee flexion on the hemiparetic side is 15 degrees.’
QA: ‘How would you describe this walking? <MOTIONSEQUENCE> Poor trunk rotation, upright posture, decreased arm swing on the left, good step progression, and walking speed.’
QA: ‘What is the strength and range of motion in the left knee for this walking pattern?’ <MOTIONSEQUENCE> The left knee has a MRC strength of ⅘ and modified Ashworth of 2.’ (Note: this data is available for the clinical gait laboratory dataset)
QA: ‘What changed between these two walking samples?’ <MOTIONSEQUENCE>, <MOTIONSEQUENCE>? Their cadence and walking speed increased, and their gait became more symmetrical with improvement in right knee flexion during swing.’
Generative motion: ‘Could you show me an example of a left hemiplegic gait with circumduction and plantarflexion? <MOTIONSEQUENCE>.’
Generative motion: ‘Could you show me an example of an able-bodied participant performing a timedup-and-go test? <MOTIONSEQUENCE>.’
Generative motion: ‘Could you show me an example of a lower-limb prosthesis user (left transfemoral amputee) performing the four-squared-step test? <MOTIONSEQUENCE>.’

The model training dataset may also include paired annotations from HumanML3D64 and KITT69.

### Methods

Tokenizer Training: In one aspect, the present method includes training a tokenizer on clinically-representative movement sequences from the dataset. State-of-the-art decoder-only language models output probability distributions over discrete tokens, which are then sampled and used in autoregressive generation. Thus, the tokens correspond to discrete entities rather than a continuous embedding, which is accomplished by training a VQ-VAE model. This autoencoder takes in an SMPL sequence over a window length of l=4 frames (captured at 12.5 fps, corresponding to 300 ms windows) and embeds into a latent token using a learned codebook.

Instead of prior approaches encoding non-physiologic joint angles from SMPL, the present method uses joint angles for the biomechanical models. The dynamic representation the FMGA system 100 produces—the joint angles with joint velocities and actions, as well as the ground reaction forces—is encoded where each time point is xₜ={θₜ, [symbol] ₜ, aₜ, gₜ} and the sequence encoded is X={xₜ, xₜ₊₁, . . . , x_{t+T-1}}. Only the velocity of the root location and heading are encoded and the absolute position is excluded, so that the representation is invariant to these and use the capacity representing other aspects of the movement. The encoder, ε, and decoder, [symbol] , consist of a 1D CNN and upsampling CNN, respectively. The model uses an embedding dimension of 512 and codebook size of 512³⁴.

$${ℒ}_{vq} = {\underbrace{{‖ X - 𝒟 [ ℰ [ X ] ] ‖}_{2}}}_{{ℒ}_{recon}} + {\underbrace{{‖ Z - sg [ Z ] ‖}_{2}}}_{{ℒ}_{embed}} + β {\underbrace{{‖ sg [ Z ] - Z ‖}_{2}}}_{{ℒ}_{commit}} \qquad ( Eq 1 )$$

The tokenizer encoder and decoder are trained with a standard VQ-VAE loss (Eq 1), which includes a reconstruction loss, embedding loss, and commitment loss. As also shown in FIG. 10A, Z is the output of the encoder, and \hat{Z} is the nearest codebook vector. Furthermore, sg[·] is the stop gradient operation. The hyperparameter β is tuned to balance the commitment loss. In some aspects, the method also incorporates training with codebook reset and exponential moving average (EMA) of the codewords, which improves generative quality.

LLM Adapter Training: In further aspects, the method includes training a low-rank adapter (LORA) using a paired language-motion dataset. LORA is a method for fine-tuning pretrained language models by adding a low-rank adapter to the model. Adjusting only a small proportion of the parameters allows the model to learn the task-specific information from the small dataset while benefiting from the large language model's generalization capabilities. As the base LLM, the present method uses a modest-sized SOTA model like the 2B instruction tuned Gemma (e.g., Gemma 3 language model). The output of an LLM is a vector of the probability of each token in the vocabulary at each time step. The present method expands LLM outputs to include the vocabulary of movement tokens from the VQ-VAE. During LORA training, the adapted LLM of the present method is trained by maximizing the likelihood of autoregressively generating the motion sequences:

$${ℒ}_{LLM} = - {\sum }_{t = 1}^{T} log P ( {z}_{t} \vert {z}_{< t} , x ) .$$

In addition to the base language vocabulary and the tokens from the VQ-VAE tokens, the language is augmented to include a start and stop token for motion sequences, <MOTION>, </MOTION>. The detokenizer works in a standard manner for language tokens, but tokens within these blocks are passed through the VQ-VAE decoder, [symbol] , to produce movement sequences.

Alternative Approaches: In some embodiments, the system may also be pretrained on the HumanML3D and KITT datasets to provide a more diverse set of prompts from an able-bodied cohort and to adequately generalize outside the defined clinical tasks created in the aforementioned training data. The training dataset may also be extended by generating more prompts in certain classes of clinical questions not included in the paired dataset.

End-user testing, including manual human evaluations and automated language benchmarks, may also be used to refine and validate the model. For automated language benchmark, standard practice for measuring the performance of LLMs on a range of language tasks, such as perplexity and BLEU scores, may be used. Quality of the generated motion sequences may also be evaluated using the Fréchet Inception Distance (FID) and diversity scores. In human evaluations, a group of physical therapists (three, for example) may evaluate the system by first being given a high-level introduction to the system and some of the questions it is expected to be able to answer. The evaluation group will review a video clip while accessing the clinical data about that patient and then will be able to ask a question. Evaluators will rank the system language answers or generated motion sequences on a 5-point Likert scale. Systematic weaknesses that can be improved with more targeted training data may be identified.

In some examples, residual VQ-VAE approaches that encode trajectories into a sequence of quantized codes that are summed may be utilized to reduce quantization errors that may limit performance. Alternatively, an approach to “Kinematic Phrases,” which helps align the latent representation with a programmatically available set of atomic movement primitives like move the wrist away, may be utilized to optimize the VQ-VAE to capture the dynamics pertinent to clinical interpretation. In some further examples, approaches to factorize the tokenizer by body parts, to provide more granular control for modifying motion trajectories may be incorporated. These factorized representations may help the language model better capture aspects of impaired movements.

### Multi-Input Motion Tokenizer

Alternatively or additionally, FIG. 10C shows another example of the model structure for a VQ-VAE model 1000 that allows selecting a trial from the input motion dataset (e.g., input 1001). For example, the VQ-VAE architecture may be used in 1D-convolutional encoders (e.g., encoders 1002) and decoders (e.g., decoders 1004) that convert joint-angle trajectories into sequences of discrete tokens (e.g., output 1005). These tokens are taken from a codebook 1006 of K vectors, each of dimension D, that is learned during VQ-VAE training. The encoder 1002 downsamples an input sequence (input 1001) of length L by factor [symbol] , producing a latent sequence of length L/ [symbol] . During encoding, at each timestep, this latent vector is replaced by the closest codebook vector by Euclidean distance. A symmetric 1D-convolutional decoder 1004 then decodes motion from the quantized embeddings.

The tokenizer may also be designed to directly embed two different formats of data from the input 1001 (e.g., a raw biomechanical dataset and a SMPL motion dataset) into a shared latent space (e.g., latent space 1003), which gives it the ability to convert between these two formats as a side product.

As referred to herein, “biomechanical dataset” refers to a clinical motion dataset containing hours of biomechanical trajectories from multiple participants performing a range of activities, many of which are standard clinical outcome assessments for mobility. Such assessments may involve, for example, overground walking, timed-up-and-go (TUG), L-test, four-square-step test (FSST), and functional gait assessments. To ensure that the model that is being trained using this data is trained to recognize the gait of people with gait impairments, many participants had gait impairments, including lower-limb amputation and the use of prosthetics, and individuals with neurological conditions such as stroke. Participants were recorded with multiple cameras and processed with marker-less motion capture algorithms to generate the kinematic trajectories.

In some examples, the biomechanical dataset may involve trials where each trial in the dataset is a trajectory associated with body shape, pelvis position in Euclidean space, pelvis orientation in 3D space, and body joint angles, where each angle is a defined degree of freedom for a joint. In some examples, to eliminate variance unrelated to movement content, body shape, pelvis position, and pelvis rotation may be discarded, and/or the wrist flexion, deviation, and supination may be zeroed out due to these factors being less pertinent for downstream tasks. In some examples, the metatarsal joint in the foot may also be zeroed as it is less accurately tracked.

The model 1000 is leveraged based on properties of human motion data by using a loss onsite location similarity. There are two dataset-specific encoders 1002 (e.g., those labeled in FIG. 10C as “biom encoder” and “smpl encoder”) that share the last layer to embed data into the same latent space 1003, while two decoders (e.g., those labeled in FIG. 10C as “biom decoder” and “smpl decoder”) decode from latent space 1003 into their respective formats. As an example of the training process, when a motion trial using biomechanical dataset is taken as model input 1001, it goes to the encoder 1002 for biomechanical dataset and is converted to a sequence of tokens. The tokens then go through both decoders 1004. The decoder 1004 for biomechanical dataset (“biom decoder”) would reconstruct the original input, while the decoder 1004 for SMPL data (“smpl decoder”) would output a sequence representing the same motion content, but in SMPL pose parameters. Then, for the outputs 1005, the original trial of biomechanical dataset is passed through forward kinematics (FK) function of the biomechanical model to obtain ground truth site locations, and the learned SMPL trial is passed through the FK function of the SMPL body model to obtain the learned locations of SMPL vertices. When input is a SMPL trial, it is processed in the opposite way. Both FK functions are differentiable and kept frozen.

For the biomechanical model, there is a set number of marker sites as in the BML-MoVi dataset, among them a subset of body surface sites are picked that have one-to-one correspondences with SMPL vertices. Additionally, a number of custom sites are defined on the head, and their locations are then found in the model and in SMPL vertices to account for head movement. These sites are shown as “site” on FIG. 10C. The site similarity loss freezes the ground truth site location and optimize for the learned site location. As root information is discarded, zero root location and fixed root orientations are inputted to FK to align output orientations. The model also learns a 10-D body shape parameter and a 3-D location offset for SMPL, to account for the difference in default body shape and root location of these two body models. During training, each input batch mixes the biomechanical dataset and SMPL data in a certain ratio, for example 50/50.

Training minimizes a combination of loss terms as shown below in the loss equation:

$$L = {L}_{biom \_ recon} + {L}_{smpl \_ recon} + {L}_{site \_ sim} + {β}_{commit} * {L}_{commit} + {β}_{shape} * {L}_{smpl \_ shape} + {β}_{pose} * {L}_{smpl \_ pose} \qquad ( Eq 2 )$$

In Eq 2, the reconstruction losses L_{biom_recon} and L_{smpl_recon} are Smooth L1 loss, the site similarity loss L_{site_sim} and VQ commitment loss L_{commit} are L2 loss, L_{smpl_shape} is L2 regularization on SMPL shape parameter, L_{smpl_pose} is L1 regularization on SMPL pose parameter converted from the biomechanical dataset; β_{commit}=0.02, βₛₕₐₚₑ=0.001, βₚₒₛₑ=0.001. The codebook learning is facilitated with an exponential moving average update and resetting to encourage codebook vector usage. Once trained, the tokenizer is fixed, and is used to convert each trial in the dataset into a sequence of motion tokens for the multimodal dataset.

Clinical Annotation and Multimodal Dataset Creation: To train and evaluate the motion-LLM, a dataset of multimodal question-and-answer tasks is created. The dataset is shuffled before use to interleave the tasks. Regarding classification tasks, for each trial the activity being performed is known, whether the participant had a gait impairment, the impairment etiology, assistive device use, and (for prosthesis users) fall history. A set of motion assessment tasks 1050 are generated in the form of question-answer pairs such as:
Q: <motion_start> <motion_1> . . . <motion_N> <motion_end> What is the most likely diagnosis for this gait impairment?
A: Stroke.

Each biomechanical trajectory is tokenized, and multimodal training examples are generated for all applicable tasks. For example, a most likely diagnosis prompt was not created for someone without a gait impairment, or a fall history for someone where no such data was collected.

For regression tasks, some clinical assessments of movement use continuously valued measurements of some aspect of subject movement, such as the time to complete a Timed Up and Go (TUG) or Four Squared Step Test (FSST). In many trials, participants walked on an instrumented walkway that measured their walking speed and cadence. Additional prompts were created to produce measures of time, speed, or cadence for these tasks.

Also, additional set of tasks were created using available annotations to improve model motion understanding. For HumanML3D dataset, a task was created where the model is asked to generate a description of the given motion sequence. For some trials in the dataset, the start and end times of some pre-defined actions types are annotated; for HumanML3D dataset, some trials have partial-sequence descriptions with a start and end time. Action localization tasks were also created, where the model is asked to output the start and end time, duration, or the content of an activity in a whole trial.

Multimodal Finetuning: Any suitable base model may be implemented. For instance, the training protocol in the example shown in FIG. 10C follows that of MotionLLM, and Gemma 3 and MedGemma are used as the base models, with a quantized low-rank adapter (QLoRA) fitted to perform supervised fine tuning, using the Hugging-face Transformers library with 4 bit quantization. Before training, the language model vocabulary is extended to include motion tokens. Unlike MotionLLM, which fine-tunes a base-pretrained model with separate adapters for motion captioning and motion generation, the aim is to train a single model across all tasks to leverage potential positive transfer. Therefore, an instruction-tuned base model and the standard Gemma chat template may be used. In some examples, the model is not trained on multi-turn conversations, or the model does not use reinforcement learning from human feedback. Since the prompts contain the added motion tokens but are otherwise short, prompt tokens may be included as part of the loss computation to help the model to learn these new elements. Using the model as explained above, it is shown that training results of the motion tokenizer demonstrate that the two formats are successfully embedded into a shared latent space.

Tokenizer Training: The tokenizers are trained with the same latent space size (e.g., 512×512) either on both datasets or on the biomechanical dataset alone, the latter using a single encoder-decoder pair and no SMPL-related losses. The reconstruction loss of biomechanical dataset is shown to converge earlier than the SMPL reconstruction loss and the site location similarity loss. As the target downstream tasks are based on the biomechanical dataset, checkpoints are selected where the reconstruction loss for biomechanical dataset converges. Both training configurations achieve low reconstruction error and high codebook usage, with similar values on the biomechanical dataset, despite the fixed latent space capability and additional loss terms. In the model trained jointly on biomechanical dataset and SMPL dataset, the codebook is shared across datasets, indicating that despite differences in representation, the two datasets contain highly overlapping motion information. Passing a trial through one encoder and the other decoder produces a conversion between dataset formats.

However, if the encoder-decoder structure is removed and pose parameters are instead directly fitted for each trial, lower site-location error can be achieved at the cost of substantially longer fitting time (approximately 3.5 minutes per trial). This tokenizer approach can be extended to additional body-model-based motion datasets, provided that a correspondence or similarity metric between site locations can be defined.

According to certain embodiments disclosed herein, tokenizer training does not rely on paired sets of biomechanical data and SMPL data; the two formats are linked via matched key points from the body-model definitions. In this case, format unification is facilitated via site alignment, referring to the following site similarity loss equation (Eq 3) where N is the number of sites with one-to-one correspondence between biomechanical dataset (site_{biom}) and SMPL dataset (siteₛₘₚₗ):

$${ℒ}_{site \_ sim} = \frac{1}{3 N} {‖ {site}_{biom} - {site}_{smpl} ‖}_{2}^{2} \qquad ( Eq 3 )$$

Each tokenizer training sample is single-format (either biomechanical or SMPL). For biomechanical input X_{biom}, the biomechanical encoder produces a token sequence, which is then consumed by both decoders: the biomechanical decoder reconstructs \hat{X}_{biom}, and the SMPL decoder outputs the same motion content in SMPL format \hat{X}ₛₘₚₗ. The site similarity loss compares ground truth biomechanical site locations to converted SMPL site locations. With forward kinematics FK_{biom}(·) and FKₛₘₚₗ(·), site_{biom} would be defined as site_{biom}=sg(FK_{biom}(X_{biom})), and siteₛₘₚₗ would be defined as siteₛₘₚₗ=FKₛₘₚₗ(\hat{X}ₛₘₚₗ), where sg(·) denotes stop-gradient. The process is symmetric for SMPL inputs. This enforces a shared discrete latent space as the same token decode consistently into both formats, thus the latent captures shared motion content regardless of the specific format. There is no separate token sets for biomechanical data versus SMPL data, and both formats map to the same token vocabulary, enabling annotation and task transfer across datasets. Training with body model swap is not applicable, as the language model is trained on token sequences (not tokenizer reconstructions); the differences between the biomechanical data and the SMPL data arise only in tokenizer reconstruction pathways, not in the token vocabulary. This approach depends only on site correspondences and naturally extends to other formats where they exist (e.g., SMPLX).

Regarding multi-task training, the language model is trained with multi-task instruction-tuning, where the dataset is a mixture of a plurality of tasks, and each model variant is trained as one single model on multiple selected tasks (not one model per task). Models trained with increasing number of tasks show an increase in performance, indicating positive transfer learning.

Regarding other loss terms, the tokenizer losses/weights follow common recipes for training VQ-VAE (e.g., the loss used in the model disclosed herein). Format-matching terms/weights are selected through experiments.

Regarding tokenizer selection, different tokenizer variants (including joint set, velocity input, masked VQ-VAE, residual VQ-VAE, codebook size, learning rate, etc.) are considered for the presently disclosed model, but no significant impact is observed on reconstruction quality (except for varying codebook size) or task performance.

Regarding transformer baselines, task-specific transformer baselines are trained for activity/impairment/diagnosis tasks. In some examples, a 6-layer encoder (e.g., 4 heads, hidden 256) is used on motion tokens, and [CLS] token embedding is used with linear head and cross-entropy, selecting the checkpoint with best F1.

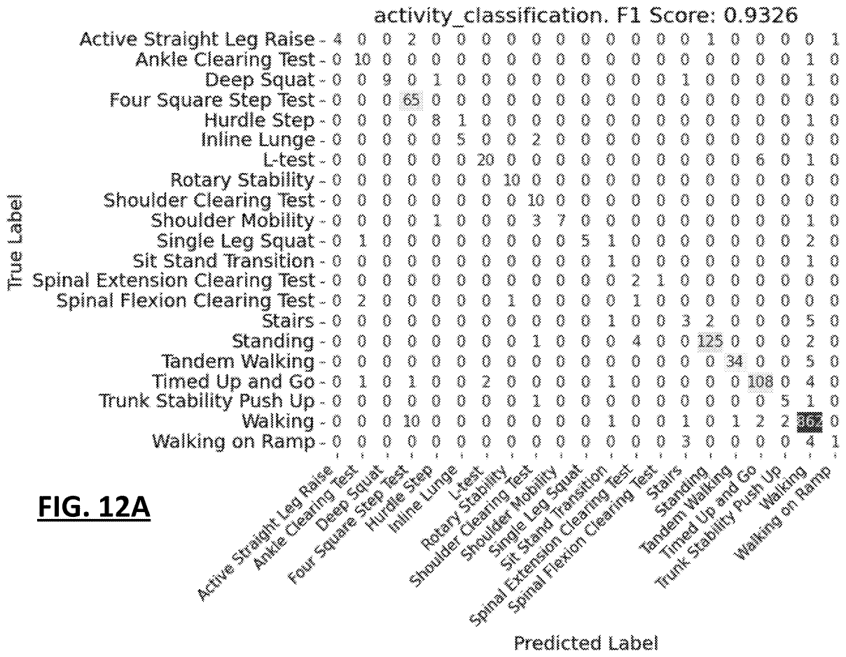

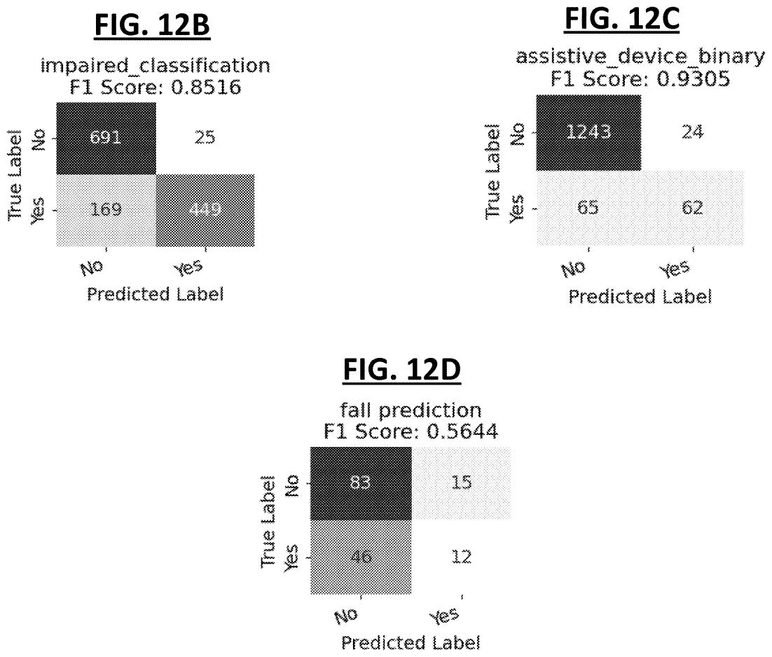

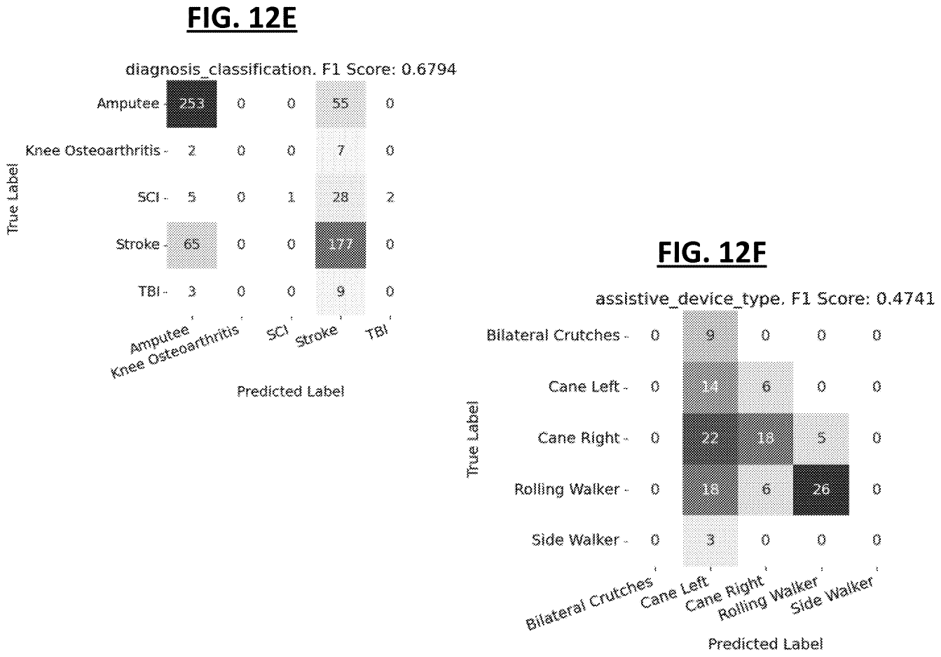

Classification Tasks and Accuracy: FIG. 12A shows a confusion matrix for activity classification, or more specifically, performance of all models on the classification tasks associated with the model 1000 of FIG. 10C. Specifically, FIG. 12A is a confusion matrix for activity classification with an F1 score of 0.93. This figure also shows the set of activities included. A confusion matrix is a performance evaluation tool used in machine learning and artificial intelligence applications, including LLMs. The confusion matrix provides a tabular representation that summarizes the performance of a classification algorithm by comparing predicted classifications against actual classifications.

In a binary classification context, as shown in FIGS. 12B and 12C, the confusion matrix comprises four categories of outcomes. True positives (“YES” for both True and Predicted) represent instances where the model correctly predicts the positive class. True negatives (“NO” for both True and Predicted) represent instances where the model correctly predicts the negative class. False positives (Predicted “YES” but True “NO”) represent instances where the model incorrectly predicts the positive class when the actual class is negative. False negatives (Predicted “NO” but True “YES”) represent instances where the model incorrectly predicts the negative class when the actual class is positive.

FIGS. 12A through 12F are exemplary confusion matrices used for analyzing the foundation model (e.g., for activity classification in FIG. 12A, impairment classification in FIG. 12B, assistive device detection in FIG. 12C, fall prediction in FIG. 12D, diagnosis classification in FIG. 12E, and assistive device type determination in FIG. 12F). For multi-class classification problems, the confusion matrix extends to an n-by-n matrix (or table), where n represents the number of distinct classes. Each row of the matrix corresponds to instances of an actual class, while each column corresponds to instances of a predicted class. The diagonal elements of the matrix represent correct predictions, while off-diagonal elements represent misclassifications.

As shown by the confusion matrices, the accuracy is notable given the substantial class imbalance in the dataset and the presence of activities with highly similar postures, such as shoulder clearing versus shoulder mobility. For impairment classification and assistive device detection, the model performed well in determining whether an individual had a movement impairment. Among participants with impairments, misclassifications were more frequent for less represented diagnoses, including spinal cord injury (SCI), traumatic brain injury (TBI), and knee osteoarthritis, consistent with class imbalance effects (FIG. 4). Activity classification yielded an F1 score of 0.9326 in FIG. 12A.

Regression Tasks: Regression tasks included four tasks with numerical outputs. They are estimating the cadence and average walking speed for trials performed on an instrumented walkway, and the manually measured time for the timed-up-and-go and the four-square-step test. These external measurements provide ground-truth walking features that are independent of the motion-capture system. To evaluate model performance on each task, the numerical output is extracted from the model's responses to task prompts, and reporting the Pearson's correlation between ground truth and predicted values. For the model, with regard to regression tasks, the correlations are high and regression slopes were highly significant (e.g., correlation of 0.98 for cadence prediction, 0.97 for speed prediction, 0.95 for TUG time prediction, 0.91 for FSST time prediction; all p<1e-10). This result shows that even if the model receives only motion tokens as input, the model's performance is highly accurate, since the answers are apparent from the raw biomechanical dataset rather than outcomes assessments that rely on a clinician's subjective impressions. As such, the model can perform well on such tasks even without additional strategies like reinforcement learning.

Model and Task Variations: For LLM base model variations, the model was trained using three base models: Gemma-3 with 4B parameters, Gemma-3 with 1B parameters, and MedGemma (a 4B model additionally fine-tuned on medical text and images). The performance scaled modestly with base model size: the 1B model achieved strong but generally reduced performance compared to the 4B model. Notably, MedGemma outperformed the 4B base model in several classification tasks and in all regression tasks, suggesting that prior medical-domain adaptation provides a beneficial base for biomechanics-language tasks.

Regarding positive transfer learning, to evaluate whether the model exhibits positive transfer learning across tasks, performance was quantified as the set of tasks included during training were varied. The 4B and 1B base models were trained on four progressively larger task sets: (1) two core tasks (activity classification and impairment classification); (2) the same two tasks plus four HumanML3D tasks; (3) all 15 clinical tasks; and (4) the full set of 19 tasks. It was observed that incorporating more tasks and thus more training data generally improved performance for both classification and regression tasks, as reflected in the confusion matrices of FIGS. 12A through 12F. This provides evidence of positive transfer in the model, where adding additional tasks enhances performance on the base tasks. For classification tasks, adding data from either related tasks within the same domain or from different sources and less closely related tasks can promote positive transfer. The improvement is also more pronounced when the target task set is smaller and when the base model size is smaller. These findings suggest that expanding the diversity of training tasks (as well as continuing to more densely annotate the biomechanical dataset) improves performance across the board. Even data originating from different sources or associated with tasks that differ from the target task appear to contribute to better learning of the shared features of human movement, thereby enhancing generalization.

Regarding unseen tasks, a model responds better for clinically relevant tasks when the model is fine-tuned on domain-specific clinical data, as compared to a model that is trained on general-purpose motion datasets.

Regarding motion description, human movement exhibits variability even within the same task, and this variability is further amplified in participants with movement impairments. In clinical datasets, annotations are limited to activity type and range action types drawn from a list, which does not necessarily capture the nuanced motion features relevant to specific tasks or individual participants. Consequently, when prompted to describe a trial, the model can only produce terms from this limited annotation set. Incorporating HumanML3D dataset which is annotated with rich natural language descriptions beneficially enables the model to generate more detailed and naturalistic descriptions for the clinical dataset as well. This enriched descriptive ability may help the model develop a deeper understanding of the underlying motion features.

In summary, improvements in motion-language model can be effectively translated to clinical movement analysis. When trained on a large and heterogeneous clinical biomechanics dataset and powered by a multi-input tokenizer that unifies disparate motion representations, the model is shown to achieve strong performance across a range of clinically meaningful tasks. Incorporating general motion-language data improves clinical task performance, which indicates that such datasets provide useful priors for clinical understanding of human movement. For this purpose, the tokenizer as disclosed herein provides an efficient way for adding in human motion data from different sources. Although the dataset is large for clinical biomechanics, it is also possible to expand task diversity and dataset breadth to further enhance the model's capability, leading to a foundation model for clinically meaningful movement with respect to accessible marker-less motion capture technology, particularly in facilitating a large biomechanics-language model as a generalist tool for movement analysis.

The system provided herein is configured to allow routine, precise, and quantitative analysis of gait and allow better monitoring of patients and their responses to interventions. The above-described model detects and temporally localizes important rehabilitation-related activities within videos, such as walking or performing clinical assessments. It also computes the ground reaction forces and joint torques to better understand foot contacts and neural control of movement. Finally, the natural language interface supports a wide range of open-ended tasks. In practice, the system may be used to improve clinical outcomes. In one aspect, the system may be used to screen gait parameters and their variability associated with fall risk during clinic visits and using these to identify people at high risk and initiate early interventions. In another aspect, the system may be used to measure ground reaction forces and knee hyperextension after spasticity interventions and using this to optimize treatment. Notably, the system is configured to function well on a diverse population, including those with developmental or acquired neurologic conditions. Including these populations, who likely are posed to get the most benefit from quantitative movement analysis, may advance fairness of artificial intelligence for people with disabilities, a critically understudied topic. The system may also be used beyond clinical gait analysis, such as for sports biomechanics, and reducing injury risks.

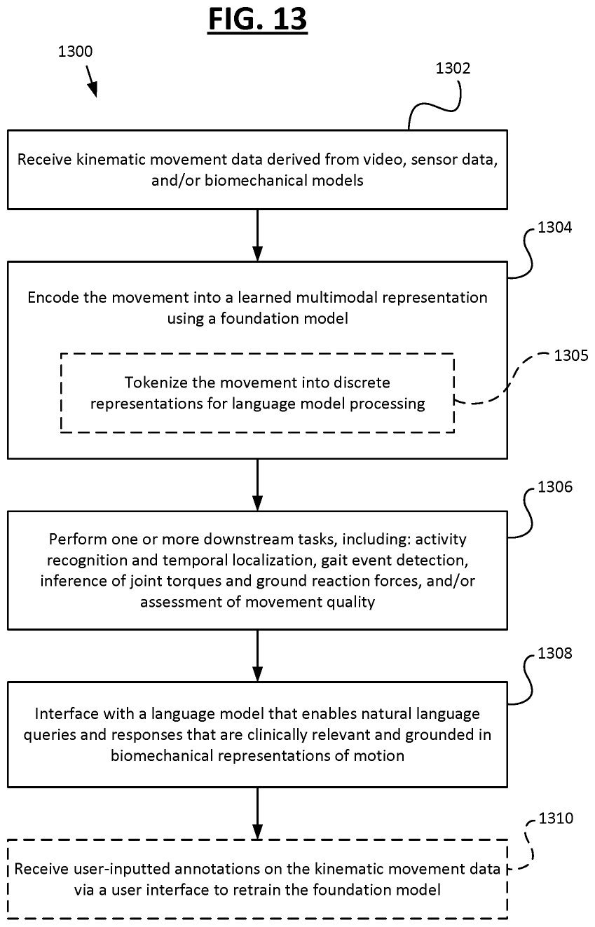

FIG. 13 shows a flowchart for a method 1300 of analyzing movements of at least one user, according to embodiments disclosed herein. Particularly, the method 1300 is incorporated using multimodal foundation models that integrate biomechanical representations of movement with machine learning and natural language processing to perform clinical gait analysis, activity recognition, kinetic inference, and clinician-facing language interaction.

In step 1302, the system or the foundation model receives kinematic movement data derived from video, sensor data, and/or biomechanical models. In step 1304, the system encodes the movement into a learned multimodal representation using a foundation model. In step 1306, the system performs one or more downstream tasks. Such tasks may include, for example, activity recognition and temporal localization, gait event detection, inference of joint torques and ground reaction forces, and/or assessment of movement quality. The tasks may be performed by decoding the learned multimodal representation using one or more task-specific decoders. In step 1308, the system interfaces with a language model that enables natural language queries and responses that are clinically relevant and grounded in biomechanical representations of motion. In some embodiments, the movement is tokenized into discrete representations that can be processed by a language model, such as in step 1305, which may be incorporated in the step 1304 or in a separate step so as to tokenize the movement into discrete representations for language model processing.

For example, the encoding performed in step 1304 may be implemented using neural network architecture such as self-supervised learning, such as the SSL pipeline 116 of FIG. 5A or the VQ-VAE model of FIG. 10A. SSL is used on gait datasets such as gait-based biomarkers to learn a representation of gait patterns that contains clinically meaningful information, for example using a combination of contrastive learning with temporal crops from a single gait trial was used as positive examples and crops from other trials as negative examples.

Regarding step 1305, in some examples, as explained with respect to FIG. 7D, a sequence of poses is used as inputs (input modality 701) for the encoding may be projected into tokens passed through several layers of a bidirectional transformer (backbone 704) to produce a series of output tokens (shared, learned multimodal representation 705), and the output tokens may be decoded (using decoder 706) into final, interpretable outputs. Such outputs may include, for example, joint angles, joint velocities, actions with the corresponding torques and ground reaction forces, fine-grained and localized activities being performed, and/or temporal localization. The transformer may also output a token for movement quality, following the SSL biomarker methodology. In some examples, tokenization of kinematic movement data into discrete representations that can be processed by a language model increases the accuracy of the language model in responding to users' natural language queries (such as those received in step 1308) as compared to providing the movement data directly (e.g., by inputting the video data), because, as explained with respect to FIGS. 8A through 8D, the model may be prone to incorrectly categorizing the content of the video data (such as categorizing a person as standing when the person is actually walking with assistance of a walker, for example). Therefore, the tokenization process beneficially reduces such mis-categorization by passing the video data through layers of a bidirectional transformer to produce a series of output tokens to be decoded.

For example, in step 1306, joint torques and ground reaction forces can be computed from the kinematic movement data. Measuring such forces allows for obtaining a deeper understanding of the gait impairments of individuals. In some examples, ground reaction forces are measured to understand how the foot makes contact with the ground relative to the rest of the body for the purpose of guiding the prescription of orthoses. Furthermore, the kinematic movement data can be used to track foot ground contacts so as to divide gait into cycles in order to measure spatiotemporal gait parameters, as part of gait event detection or movement quality assessment. In another aspect, the foundation model is also trained to infer joint torques and ground reaction forces from kinematics by incorporating self-supervised learning with biomechanical physics simulation into the training process using, for example, any suitable (e.g., high-performance and GPU-accelerated) differentiable biomechanical simulator.

For example, in step 1308, the language model may be capable of receiving natural language queries and respond with clinically relevant answers that are grounded in biomechanical representation of human body's motion. As shown in FIG. 11, this may be facilitated via the use of a user interface 204 that has an input section 1100 for the user to input natural language queries and expect a response from the language model that is accurate in view of the multimodal foundation models that are integrated with biomechanical representations of movement with machine learning and natural language processing.

In some examples, as shown in step 1310, the foundation model may receive user-inputted annotations regarding the kinematic movement data. This may be provided via a user interface, such as the user interface 204 shown in FIG. 6, where the user may give a rating on numerous factors associated with the kinematic movement data, such as the visibility, accuracy of the bounding box tracking, and/or the accuracy of the key point tracking. Accordingly, the foundation model may be retrained based on the user annotation so as to further improve the accuracy of the foundation model in future inferences and iterations.

A number of implementations have been described. Nevertheless, it will be understood that various modifications may be made without departing from the spirit and scope of the disclosure. Accordingly, other implementations are within the scope of the following claims.

1. A method for analyzing movement of at least one user, the method comprising:
receiving kinematic movement data derived from one or more of: video data, sensor data, or biomechanical models;
encoding the kinematic movement data into a learned multimodal representation using a foundation model; and
performing, using the learned multimodal representation, one or more downstream tasks by decoding the learned multimodal representation using one or more task-specific decoder.

2. The method of claim 1, further comprising:
interfacing with a language model that enables natural language queries and responses that are clinically relevant and grounded in biomechanical representations of motion.

3. The method of claim 1, wherein the one or more downstream tasks include at least one of: activity recognition and temporal localization, gait event detection, inference of joint torques and ground reaction forces, or assessment of movement quality.

4. The method of claim 1, wherein the foundation model is a self-supervised machine learning model that is trained on training data that includes a plurality of movement impairments.

5. The method of claim 1, further comprising:
receiving, from a user interface, user annotations associated with the one or more downstream tasks; and
retraining the foundation model using the user annotations.

6. The method of claim 1, wherein the encoding the kinematic movement data into the learned multimodal representation comprises tokenizing the kinematic movement data into a plurality of discrete representations that can be processed by a language model.

7. The method of claim 6, wherein the tokenizing is facilitated by a tokenizer that is trained on a plurality of clinically-representative movement sequences.

8. The method of claim 7, wherein the tokenizer comprises a vector-quantized variational autoencoder.

9. The method of claim 1, wherein the foundation model is trained using a dataset of paired motion and language data associated with a plurality of data subjects.

10. The method of claim 9, wherein the dataset of paired motion and language data comprises one or more of:
a) demographic information, primary rehabilitation diagnosis, and comorbidities associated with the plurality of data subjects;
b) annotations for activities, assistive devices being used, and movement scores, such as the time for a timed-up-and-go, associated with movement video data of the plurality of data subjects;
c) walking features, such as spatiotemporal gait parameters, joint range of motion, and movement symmetry associated with the plurality of data subjects;
d) performance on a plurality of clinical outcome assessments performed as part of routine care for the plurality of data subjects; and
e) one or more inputs from an annotation dashboard on a user interface, the inputs including at least one clinical impression of movement that is transcribed and stored in the dataset.

11. A foundation model system for analyzing movement of at least one user, the system comprising:
a processing unit; and
a non-transitory computer-readable medium storing instructions thereon which, when executed by the processing unit, cause the processing unit to:
receive kinematic movement data derived from one or more of: video data, sensor data, or biomechanical models;
encode the kinematic movement data into a learned multimodal representation using a foundation model; and
perform, using the learned multimodal representation, one or more downstream tasks by decoding the learned multimodal representation using one or more task-specific decoder.

12. The system of claim 11, wherein the instructions further cause the processing unit to:
interface with a language model that enables natural language queries and responses that are clinically relevant and grounded in biomechanical representations of motion.

13. The system of claim 11, wherein the one or more downstream tasks include at least one of: activity recognition and temporal localization, gait event detection, inference of joint torques and ground reaction forces, or assessment of movement quality.

14. The system of claim 11, wherein the foundation model is a self-supervised machine learning model that is trained on training data that includes a plurality of movement impairments.

15. The system of claim 11, wherein the instructions further cause the processing unit to:
receive, from a user interface, user annotations associated with the one or more downstream tasks; and
retrain the foundation model using the user annotations.

16. The system of claim 11, wherein the encoding the kinematic movement data into the learned multimodal representation comprises tokenizing the kinematic movement data into a plurality of discrete representations that can be processed by a language model.

17. The system of claim 16, wherein the tokenizing is facilitated by a tokenizer that is trained on a plurality of clinically-representative movement sequences.

18. The system of claim 17, wherein the tokenizer comprises a vector-quantized variational autoencoder.

19. The system of claim 11, wherein the foundation model is trained using a dataset of paired motion and language data associated with a plurality of data subjects.

20. The system of claim 19, wherein the dataset of paired motion and language data comprises one or more of:
a) demographic information, primary rehabilitation diagnosis, and comorbidities associated with the plurality of data subjects;
b) annotations for activities, assistive devices being used, and movement scores, such as the time for a timed-up-and-go, associated with movement video data of the plurality of data subjects;
c) walking features, such as spatiotemporal gait parameters, joint range of motion, and movement symmetry associated with the plurality of data subjects;
d) performance on a plurality of clinical outcome assessments performed as part of routine care for the plurality of data subjects; and
e) one or more inputs from an annotation dashboard on a user interface, the inputs including at least one clinical impression of movement that is transcribed and stored in the dataset.
